(19) United States
(12) Patent Application Publication
(10) Pub. No.: US 2026/0258117 A1
(43) Pub. Date: Sep. 3, 2026

(54) BISPECIFIC ANTIBODY AND USES THEREOF

(71) Applicant: ACADEMISCH ZIEKENHUIS LEIDEN (H.O.D.N. LUMC), Leiden (NL)

(72) Inventors: Leendert Adrianus TROUW, Leiden (NL); Fleur Suzanne VAN DE BOVENKAMP, Leiden (NL); Paul Willem Henri Ida PARREN, Leiden (NL)

(21) Appl. No.: 18/880,125

(22) PCT Filed: Jun. 21, 2023
(86) PCT No.: PCT/NL2023/050345

§ 371 Date: Dec. 30, 2024

(30) Foreign Application Priority Data
Jul. 6, 2022 (NL) 2032398

(51) Int. Cl. C07K 16/18; A61P 37/06; C07K 16/28

(52) U.S. Cl. CPC C07K 16/18; A61P 37/06; C07K 16/2833; C07K 16/2896; C07K 2317/31; C07K 2317/565; C07K 2317/76

(57) ABSTRACT

To reduce damage in complement mediated diseases, several complement inhibitory drugs have been developed, of which few have made their way to the clinic and are approved treatments. Bispecific antibodies are disclosed herein for the treatment of complement mediated diseases. Locally targeted complement inhibition is possible using a bispecific antibody (bsAb) that engages human endogenous complement inhibitors when being targeted to a specific antigen or site in the body. The present invention represents a promising novel therapeutic approach for the treatment of complement-mediated diseases. Since the bsAbs bind to local antigens and therefore result in targeted inhibition, lower dosing and thereby lower costs are additional advantages.

### BACKGROUND

The complement system is important for the clearance of pathogens, thereby providing protection against infection. It consists of three pathways, initiated by different molecules (e.g. antibodies or sugar structures on pathogens), but all resulting in the formation of a C₃ convertase. Cleavage of C3 by the C3 convertase results in the formation of a C5 convertase, which results in the formation of a membrane attack complex (C5b-9) and the anaphylatoxins C3a and C5a. These anaphylatoxins recruit and activate immune cells, which contributes to the initiation of the adaptive immune response.

Undesired or excessive complement activation contributes to cell and tissue damage. This is the case in several (auto) antibody-driven diseases that predominantly activate the classical pathway, for example in autoimmune diseases (anti-collagen antibodies in arthritis (1), anti-glomerular basement membrane antibodies in nephritis (2), anti-acetylcholine receptor antibodies in myasthenia gravis (3) and transplant rejection (anti-HLA antibodies) (4)). However, the lectin pathway and alternative pathway-driven complement activation can also result in severe damage, for example in atypical hemolytic uremic syndrome or age-related macular degeneration (5).

To reduce damage in these diseases, several complement inhibitory drugs have been developed, of which a few have made their way to the clinic and were approved for the treatment of several complement-mediated diseases. Importantly, these therapeutics inhibit complement systemically. This is associated with important downsides, including the increased risk for generalized immune suppression, infections and high dosing, therefore there is a need for new and improved methods of treating complement mediated diseases without the current severe side effects. The few complement inhibitory drugs that have made their way to the clinic and were approved for the treatment of several complement-mediated diseases inhibit complement systemically, which is associated with important downsides including the increased risk for infections and high dosing. Eculizumab (anti-C5 antibody) is an example of an FDA- and EMA-approved complement inhibitory drug to treat patients suffering from paroxysmal nocturnal hemoglobinuria, atypical hemolytic uremic syndrome, generalized myasthenia gravis, and neuromyelitis optica spectrum disorder (17). Since life-threatening and fatal meningococcal infections have occurred in patients treated with eculizumab, patients are immunized with meningococcal vaccines prior to administering eculizumab, and patients are monitored closely for early signs of meningococcal infections. Therefore, eculizumab is not broadly applicable and only used in patients with rare diseases, making this drug one of the most expensive drugs.

### BRIEF SUMMARY OF THE DISCLOSURE

The invention is based on the surprising finding that locally targeted complement inhibition is possible using a bispecific antibody (bsAb) that engages human endogenous complement inhibitors when being targeted to a specific antigen or site in the body. The present invention represents a promising novel therapeutic approach for the treatment of complement-mediated diseases. Since the bsAbs bind to local antigens and therefore result in targeted inhibition, lower dosing and thereby lower costs are additional advantages.

In a first aspect, the present invention provides a bispecific antibody comprising a first antigen binding domain that binds a complement inhibitor and a second antigen binding domain that binds a target antigen.

In another aspect, the present invention provides the bispecific antibody according to the first aspect of the invention for use in a method for treating or preventing a disease or condition in an individual.

In another aspect, the present invention provides the bispecific antibody according to the first aspect for use in the treatment of a complement mediated disease.

In another aspect, the present invention provides a bispecific antibody according to the first aspect of the invention conjugated to an additional therapeutic moiety.

In another aspect, the present invention provides a composition comprising the bispecific antibody according to the first aspect of the invention and at least one pharmaceutically acceptable diluent or carrier.

In another aspect of the present invention the bispecific antibodies according to the first aspect are typically produced recombinantly, i.e. by expression of nucleic acid constructs encoding the antibodies in suitable host cells, followed by purification of the produced recombinant antibody from the cell culture. Nucleic acid constructs can be produced by standard molecular biological techniques well-known in the art. The constructs are typically introduced into the host cell using an expression vector. Suitable nucleic acid constructs and expression vectors are known in the art. Host cells suitable for the recombinant expression of antibodies are well-known in the art, and include CHO, HEK-293, Expi293F, PER-C6, NS/0 and Sp2/0 cells. Expression may also be performed in yeast (e.g. Pichia pastoris) or bacteria (e.g. Escherichia coli).

Accordingly, in a further aspect, the invention relates to a nucleic acid construct encoding a bispecific antibody according to the first aspect of the invention. In one embodiment, the construct is a DNA construct. In another embodiment, the construct is an RNA construct. In a further aspect, the invention relates to an expression vector comprising a nucleic acid construct encoding a bispecific antibody according to the first aspect of the invention. In a further aspect, the invention relates to a host cell comprising one or more nucleic acid constructs encoding a bispecific antibody according to the first aspect of the invention or an expression vector comprising a nucleic acid construct encoding a bispecific antibody according to the first aspect of the invention.

In a further aspect, the invention relates to a process for manufacturing the bispecific antibody according to the first aspect of the invention, comprising expressing one or more nucleic acids encoding the bispecific antibody according to the first aspect of the invention in a host cell. In a further aspect, the invention the relates to a process for manufacturing a clinical batch of the bispecific antibody according to a first aspect of the invention, comprising expressing one or more nucleic acids encoding the bispecific antibody according to the first aspect of the invention in a host cell. A “clinical batch” when used herein refers to a product composition that is suitable for use in humans.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows successful generation of bispecific antibodies. (A) BsAbs were tested for binding to both antigens in a bispecific ELISA. Plates were coated with BSA-DNP and incubated with anti-FH (upper graph, diamonds), anti-C4BP (lower graph, diamonds), anti-DNP (hexagons) parental Abs, or bsAbs (circles). FH or C4BP were added and these inhibitors were detected. (B) Masses of parental and bsAbs were determined by mass spectrometry.

FIG. 2 shows targeted bispecific antibodies that bind endogenous complement inhibitors decrease complement activation in situ. BsAbs were functionally tested in plate-bound complement activation assays. Plates coated with BSA-DNP and IgG (classical pathway), acetylated HSA (lectin pathway), or LPS (alternative pathway) were incubated with a titration of anti-FH x anti-DNP (A,C), 5 μg/mL anti-FH x anti-DNP (B,D), a titration of anti-C4BP x anti-DNP (E,G), or 5 μg/mL anti-C4BP x anti-DNP (F,H) or control bsAbs, with NHS with (+) or without (−) exogenous FH (A-D) or C4BP (E-H). FH (A,B), C4BP (E,F), and C5b-9 (C,D,G,H) were detected. Bars indicate means and error bars indicate standard deviations. One-way ANOVA compared to no bsAb, ** P<0.01; *** P<0.001; **** P<0.0001. Anti-HIV is used as an irrelevant binding control.

FIG. 3 shows targeted bispecific antibodies that bind endogenous complement inhibitors decrease IgM-initiated complement activation. BsAbs were functionally tested in plate-bound complement activation assays. Plates coated with BSA-DNP and IgM were incubated with a titration of anti-FH x anti-DNP (A), 5 g/mL anti-FH x anti-DNP (B), a titration of anti-C4BP x anti-DNP (C), or 5 μg/mL anti-C4BP x anti-DNP (D) or control bsAbs, with NHS with (+) or without (−) exogenous FH (B) or C4BP (D). FH (A,B), C4BP (C,D), and C5b-9 (A-D) were detected. Bars indicate means and error bars indicate standard deviations. One-way ANOVA compared to no bsAb, ** P<0.01; *** P<0.001; **** P<0.0001.

FIG. 4 shows targeted bispecific antibodies that bind endogenous complement inhibitors decrease lectin pathway-initiated complement activation. BsAbs were functionally tested in plate-bound complement activation assays. Plates coated with BSA-DNP and acetylated HSA (lectin pathway) were incubated with 5 μg/mL anti-FH x anti-DNP (A) or anti-C4BP x anti-DNP (B) or control bsAbs, with Clq-depleted serum (left) or factor B-depleted serum (right) with (+) or without (−) exogenous FH (A) or C4BP (B). FH (A), C4BP (B), and C5b-9 (A,B) were detected. Bars indicate means and error bars indicate standard deviations. One-way ANOVA compared to no bsAb, ** P<0.01; **** P<0.0001.

FIG. 5 shows biotin-targeting bispecific antibodies that bind endogenous complement inhibitors decrease complement activation. (A) BsAbs were tested for binding to both antigens in a bispecific ELISA. Plates were coated with biotinylated BSA-DNP and incubated with anti-FH (top figure, diamonds), anti-C4BP (bottom figure, diamonds), anti-biotin (hexagons) parental Abs, or bsAbs (circles). FH or C4BP was added and these inhibitors were detected. (B) BsAbs were functionally tested in plate-bound complement activation assays. Plates coated with biotinylated BSA-DNP and IgG (classical pathway) were incubated with 5 μg/mL anti-FH x anti-biotin (B) or anti-C4BP x anti-biotin (C) or control bsAbs, with NHS with (+) or without (−) exogenous FH (B) or C4BP (C). FH (B), C4BP (C), and C5b-9 (B,C) were detected. Bars indicate means and error bars indicate standard deviations. One-way ANOVA compared to no bsAb, ** P<0.01; **** P<0.0001.

FIG. 6 shows targeted bispecific antibodies decrease lysis of liposomes. The effect of the bsAbs on the classical pathway was analyzed in a fluorescence-based complement activation assay using liposomes. Liposomes containing DNP and CD52, filled with sulforhodamine B, were incubated with anti-FH x anti-DNP (A) or anti-C4BP x anti-DNP (B) or control bsAbs, with (+, bottom figure) or without (−, top figure) exogenous FH/C4BP and NHS. After 1 hour, sulforhodamine B fluorescence was measured. After 10 minutes (0 minutes in graphs), anti-CD52 wild-type was added and fluorescence was measured for another 30 minutes.

FIG. 7 shows targeted bispecific antibodies protect erythrocytes from complement-mediated lysis. The effect of the targeted bsAbs on activity of the classical pathway was analyzed in a hemolytic assay. Biotinylated antibody-sensitized sheep red blood cells were incubated with anti-FH x anti-biotin (A) or anti-C4BP x anti-biotin (B) or control bsAbs, with (+) or without (−) exogenous FH/C4BP. Subsequently, cells were incubated with 0.5% NHS and centrifuged, and supernatants were measured. The effect of the targeted bsAbs on activity of the alternative pathway was analyzed in a similar assay. Biotinylated rabbit red blood cells were incubated with anti-FH x anti-biotin (A) or anti-C4BP x anti-biotin (B) or control bsAbs, with (+) or without (−) exogenous FH/C4BP. Subsequently, cells were incubated with 5% NHS and centrifuged, and supernatants were measured. Bars indicate means and error bars indicate standard deviations. One-way ANOVA compared to no bsAb, *** P<0.001; **** P<0.0001.

FIG. 8 shows targeted bispecific antibodies protect white blood cells from complement-mediated cytotoxicity. BsAbs were functionally tested in a complement-dependent cytotoxicity assay. (A) Ramos cells were incubated with anti-CD20 wild-type with or without anti-FH x anti-HLA I or control bsAbs, with (+) or without (−) exogenous FH. Cells were washed and incubated with 10% NHS. FH and cell viability (using 7-AAD) were measured. (B) PBMCs were incubated with anti-CD52 wild-type with or without anti-FH x anti-HLA I or control bsAbs, with (+) or without (−) exogenous FH. Cells were washed and incubated with 10% NHS. FH and cell viability (using 7-AAD) were measured. Bars indicate means and error bars indicate standard deviations. One-way ANOVA compared to no bsAb, ** P<0.01; *** P<0.001; **** P<0.0001.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

A first aspect of the invention is a bispecific antibody comprising a first antigen binding domain that binds a complement inhibitor and a second antigen binding domain that binds a target antigen.

As used herein, a “complement inhibitor” is a molecule that prevents or reduces activation and/or propagation of the complement cascade that results in the formation of C5a or prevents binding of complement effectors to their receptors. A complement inhibitor can operate on one or more of the complement pathways, i.e., classical, alternative or lectin pathway.

Complement inhibitors target different levels and steps of the complement cascade. The complement inhibitor can be one or more of a C5a inhibitor, an inhibitor of the receptors for C5a (C5aR1/C5aR2), a C3a inhibitor, an inhibitor of the receptor for C3a (C3aR), a factor D inhibitor, a factor B inhibitor, a factor P inhibitor, a FHR-1 inhibitor, a FHR-2 inhibitor, a FHR-3 inhibitor, a FHR-4 inhibitor, a FHR-5 inhibitor, a Clq inhibitor, a C2 inhibitor, C3 inhibitor, C4 inhibitor, a C5 inhibitor, a C6 inhibitor, a C7 inhibitor, a C8 inhibitor, a C9 inhibitor, a MBL inhibitor, a MASP1 inhibitor, a MASP2 inhibitor, a MASP3 inhibitor, a Ficolin1 inhibitor, a Ficolin2 inhibitor, a Ficolin3 inhibitor, a Collectin10 inhibitor, a Collectin11 inhibitor, an inhibitor of the complement fragments as present in the C3-convertases or C5-convertasessuch as C3b, C4b, Bb, C2a and the convertases C4b2a, C3bBb, C3bBbP, C4b2aC3b, C3bBbC3b or can be FH, mini-FH, FHL-1, C4BP, C1-INH, complement receptor 1 (CR1, CD35), membrane cofactor protein (MCP, CD46), decay-accelerating factor (DAF, CD55), CD59,, and factor I (FI) MAp44 (MBL-associated protein 1; MAP-1) and small MBL-associated protein (sMAP; also termed MAp19 or MAP-2), truncated or mini-versions of the complement proteins or any combination thereof.

As used herein, a “target antigen”, is an antigen that attracts the bispecific antibody to the location of choice within the body. In a preferred embodiment the location of choice is the location wherein the antibody is most effective at treating a complement mediated disease. In one embodiment the bispecific antibody is a bispecific antibody according to the first aspect, wherein both antigen binding domains bind human endogenous antigens. As used herein, a “human endogenous antigen”, is an antigen that originates from within the human.

“Inhibit complement activation locally” refers to the inhibition of complement activation that occurs outside the vascular system. The present invention is based in part on the inventors' recognition that (i) soluble complement proteins are produced locally in a variety of disorders; (ii) most or all systemic complement proteins do not readily extravasate and participate in complement activation outside of the vasculature unless there has been damage to the vasculature, and (iii) activation of locally produced complement proteins, including locally produced soluble complement proteins, plays a significant role in disease pathology and symptoms, often exceeding the role played by activation of systemic complement. Local complement activation involving locally produced soluble complement proteins frequently takes place in bodily compartments.

In another embodiment the complement inhibitor is selected from the group consisting of: the endogenous complement regulator factor H (FH), C4b-binding protein (C4BP), complement receptor 1 (CR1, CD35), membrane cofactor protein (MCP, CD46), decay-accelerating factor (DAF, CD55), CD59, FHL-1, mini-FH, factor I (FI) MAp44 (MBL-associated protein 1; MAP-1) and small MBL-associated protein (sMAP; also termed MAp19 or MAP-2).

In another embodiment the complement inhibitor is C4BP or FH. In another embodiment the complement inhibitor is C4BP. In another embodiment the complement inhibitor is FH.

“Complement Factor H protein”, “FH”, “CFH protein”, or “CFH” as used herein refers to a protein of approximately 150 kDa (UniProt P08603) that is a member of the regulators of complement activation family and is a complement control protein. CFH is a large soluble glycoprotein that circulates in human plasma and serves to regulate the alternative pathway of the complement system, ensuring that the complement system is directed towards pathogens or other dangerous material and does not damage host tissue. CFH is a cofactor in the inactivation of C3b by factor I and functions to increase the rate of dissociation of the C3bBb complex (C3 convertase) and the C₃bBbC3b complex (C5 convertase) in the alternative complement pathway. CFH binds to glycosaminoglycans that are generally present on host cells but not, normally, on pathogen surfaces. CFH is composed of 20 short consensus repeats (SCRs), some of which function in cell attachment, while others function to eliminate C3b from the cell surface. The 20 SCRs that comprise CFH are each approximately 60 amino acids long, are arranged head to tail, and contain 4 cysteine residues forming 2 disulfide bonds per module. The C3b binding domain may refer to the part of the CFH that binds to C3b. SCRs 19 and 20 are involved in cellular binding.

Complement C4b binding protein (C4BP) is a multiple polypeptide chain plasma glycoprotein. The protein is composed of seven identical alfa-chains and one beta chain linked by their C-terminal parts in a central core. It inhibits the action of C4b in the C3-convertase C4b2a. It splits C4b in the convertase and by acting as a cofactor for factor I which cleaves C4b. C4BP binds necrotic cells and DNA, to clean up after swelling. C4BP predominantly binds C4b, via SCR 1-3 of the alpha chains.

Surprisingly the bispecific antibodies of the present invention can bind to, retain or capture the circulating complement inhibitors and transport the complement inhibitor to the location of interest without affecting the activity of the complement inhibitor. The activity of the bispecific antibody bound complement inhibitors of the present invention is unaffected, more preferably is superior to unbound complement inhibitors. The endogenous complement inhibitors FH and C4BP retain their complement inhibitor function while being bound to the bispecific antibody of the present invention.

In another embodiment the first antigen binding domain inhibits complement protein function. Preferably the first antigen binding domain inhibits C1s or C5 complement function. In another embodiment the target antigen is a tissue specific antigen. The target antigen may be a complement molecule, preferably C1s or C5. In a further embodiment the target antigen is an autoantigen. The target antigen may be selected from the group consisting of: Gangliosides, Acetylcholine Receptor/MUSK, Aquaporins, Amyloid Beta, TF, VCAM, NC1 domain of collagen type IV, Clq, MPO, PR3, TF, Collagen, PTM-proteins, Desmogleins, HLA-I/HLA-II antigens, and C3d.

In a further embodiment the bispecific antibody of the first aspect of the invention further comprises a mutation in the Fc region, preferably the mutation comprises an L234A, L235A (LALA) mutation and/or an L234A, L235A, P329G (LALAPG) mutation in the Fc region. The mutation in the Fc region may abolish the interaction between the Fc region and FcRs as well as abolish interaction with Clq.

In the context of bispecific antibodies of the present invention, the multimerizing domains, e.g., Fc domains, may comprise one or more amino acid changes (e.g., insertions, deletions or substitutions) as compared to the wild-type, naturally occurring version of the Fc domain. For example, the invention includes bispecific antibodies comprising one or more modifications in the Fc domain that results in a modified Fc domain having a modified (pH-dependent) binding interaction (e.g., enhanced or diminished) between Fc and FcRn. Possible Fc modifications include an LALAPG mutation, LFLEPG mutation and LFLEDA mutation (L234A-L235A-P329G) (L234F-L235E-P329G, L234F-L235E-D265A). Alternatively this can include LALA or LELE mutations (L234A-L235A), (L235E-L235E).

In some embodiments, the first and/or second Fc polypeptides contain mutations that render the antibody inert, i.e. unable to, or having reduced ability to, mediate effector functions. In one embodiment, the inert Fc region is in addition not able to bind Clq. In one embodiment, the first and second Fc polypeptides comprise a mutation at position 234 and/or 235, preferably the first and second Fc polypeptide comprise an L234F and an L235E substitution, wherein the amino acid positions correspond to human IgG1 according to the EU numbering system. In another embodiment, the antibody contains a L234A mutation, a L235A mutation and a P329G mutation. In another embodiment, the antibody contains a L234F mutation, a L235E mutation and a D265A mutation.

In certain embodiments, the Fc domain may be a combination of multiple Fc sequences derived from more than one immunoglobulin isotype.

In another embodiment the bispecific antibody according to the first aspect of the invention binds endogenous complement inhibitors and reduces complement activation compared to an untreated subject.

As used herein, “reduces complement activation” is measured as a reduced incorporation of the membrane attack complex and/or decreased of C3b. In an embodiment the reduction of complement activation may be a 5% reduction compared to no treatment, furthermore an embodiment of the invention may be 10%, 15%, 20%, 25%, 30%, 35%, 40%, 45%, a greater than 50% reduction in complement activation.

In another embodiment the bispecific antibody according to the first aspect of the invention binds endogenous complement inhibitors and decreases complement mediated lysis compared to an untreated subject. The decrease of complement mediated lysis may be a decrease in cell death compared to an untreated subject. The decrease of complement mediated lysis may be a decrease in cell death compared to an untreated subject by at least 10%, e.g. between 10-20%, 20-30%, 30-40%, 40-50%, 50-60%, 60-70%, 70-80%, or greater than 80% reduction, greater than 90% reduction or a 100% reduction.

As used herein “decreases complement mediated lysis” is measured as reduced positivity for cell death markers such as Propidium Iodide (PI), 7-aminoactinomycin (7AAD) or the release of Lactate Dehydrogenase (LDH).

In another embodiment the bispecific antibody inhibits complement activation locally.

In another embodiment the present invention provides a bispecific antibody, wherein the variable heavy and variable light chain comprise the heavy and light chain sequences as outlined in Table 2.

In another embodiment a bispecific antibody according to the present invention, wherein the antigen binding domain comprises:
a) a first heavy chain variable region CDR1 comprising the amino acid sequence SEQ ID NO: 15; a heavy chain variable region CDR2 comprising the amino acid sequence SEQ ID NO: 16; a heavy chain variable region CDR3 comprising the amino acid sequence SEQ ID NO: 17 and
a first light chain variable region CDR1 comprising the amino acid sequence SEQ ID NO: 18; a light chain variable region CDR2 comprising the amino acid sequence SEQ ID NO: 19, a light chain variable region CDR2 comprising the amino acid sequence SEQ ID NO: 20 or
b) a first heavy chain variable region CDR1 comprising the amino acid sequence SEQ ID NO: 21; a heavy chain variable region CDR2 comprising the amino acid sequence SEQ ID NO: 22; a heavy chain variable region CDR3 comprising the amino acid sequence SEQ ID NO: 23 and
a first light chain variable region CDR1 comprising the amino acid sequence SEQ ID NO: 24; a light chain variable region CDR2 comprising the amino acid sequence SEQ ID NO: 25, a light chain variable region CDR2 comprising the amino acid sequence SEQ ID NO: 26.

In another embodiment a bispecific antibody according to the present invention, wherein the antigen binding domain comprises:
a) a second heavy chain variable region CDR1 comprising the amino acid sequence SEQ ID NO: 15; a heavy chain variable region CDR2 comprising the amino acid sequence SEQ ID NO: 16; a heavy chain variable region CDR3 comprising the amino acid sequence SEQ ID NO: 17 and
a second light chain variable region CDR1 comprising the amino acid sequence SEQ ID NO: 18; a light chain variable region CDR2 comprising the amino acid sequence SEQ ID NO: 19, a light chain variable region CDR2 comprising the amino acid sequence SEQ ID NO: 20, or
b) a second heavy chain variable region CDR1 comprising the amino acid sequence SEQ ID NO: 21; a heavy chain variable region CDR2 comprising the amino acid sequence SEQ ID NO: 22; a heavy chain variable region CDR3 comprising the amino acid sequence SEQ ID NO: 23 and
a second light chain variable region CDR1 comprising the amino acid sequence SEQ ID NO: 24; a light chain variable region CDR2 comprising the amino acid sequence SEQ ID NO: 25, a light chain variable region CDR2 comprising the amino acid sequence SEQ ID NO: 26.

In another embodiment a bispecific antibody according to the present invention, wherein the first and/or second antigen binding site comprises a variable heavy chain comprising amino acid SEQ ID NO: 1 and a variable light chain comprising amino acid SEQ ID NO: 2 or wherein the first and/or second antigen binding site comprises a variable heavy chain comprising amino acid SEQ ID NO: 3 and a variable light chain comprising amino acid SEQ ID NO: 4. In another aspect the present invention provides a bispecific antibody according to the first aspect of the invention for use in a method for treating or preventing a disease or condition in an individual.

In another aspect the present invention provides a bispecific antibody according to the first aspect of the invention for use in the treatment of a complement mediated disease. In another embodiment an additional bispecific antibody is used in the treatment of disease. The additional bispecific antibody is a bispecific antibody according to the first aspect of the invention wherein the additional bispecific antibody binds a different complement inhibitor and/or a different target antigen. The additional bispecific antibody may also bind the same complement inhibitor and a different target antigen. The additional bispecific antibody may also bind a different complement inhibitor and the same target antigen.

The bispecific antibody of the present invention for use in the treatment of autoimmune disease, Rheumatoid arthritis, Osteo arthritis, Psoriatic arthritis, Systemic Lupus Erythematosus, Myasthenia Gravis and Pemphigus, renal disease, atypical haemolytic uremic syndrome, Membranoproliferative glomerulonephritis, IgA nephropathy, Diabetic Nephropathy, ANCA vasculitis, complement 3 glomerulonephritis, a neurological disorder, Guillain Barre Syndrome, AMAN, Miller Fisher Syndrome, Myastenia Gravis, Neuromyelitis Optica, Alzheimer's disease, Parkinson's Disease, eye disease, dry and wet age related macular degeneration, motor neuron disease, complement regulation disorder, paroxysmal nocturnal hemoglobinuria, arthritis, nephritis, transplant rejection, IBMIR, cancer, infertility, pregnancy complications or HELPP syndrome or atypical hemolytic uremic syndrome. The target antigen may be specific to the disease being treated.

The bispecific antibody for use in the treatment of diseases may target specific target antigens based on the disease to be treated as set out in the table below:

| Disease | Target antigen |
|---|---|
| Guillain Barre Syndrome, AMAN, | Gangliosides |
| Miller Fisher Syndrome | |
| Myastenia Gravis | Acetylcholine Receptor/MUSK |
| Neuromyelitis Optica | Aquaporins |
| Alzheimers Disease | Amyloid Beta |
| Dry and wet AMD | TF, VCAM |
| Anti-GBM Nephritis | NC1 domain of collagen type IV |
| MPGN I/II | C1q, TF, VCAM |
| C3G and aHUS | TF, VCAM |
| Lupus Nephritis | C1q, TF, VCAM |
| ANCA Vasculitis | MPO, PR3, TF, VCAM |
| Arthritis | Collagen, PTM-proteins |
| Pemphigus Vulgaris/Autoimmune | Desmogleins |
| Bullous Dermatoses | |
| Transplant rejection | HLA-I/HLA-II antigens, TF, VCAM |
| PNH | C3d |
| Cardiovascular disease | TF, VCAM |

As used herein, “complement mediated disease” is a disorder associated with enhanced complement activation or decreased complement inhibition. These conditions can be triggered by complement activation of any of the complement pathways individually or any combination of pathways. In addition, it may involve the complement C3b feedback cycle.

The complement-mediated disorder may be a disorder characterised by symptoms that are ameliorated by increased levels of C3b-inactivating and iC3b-degradation activity in the subject.

Examples of complement-mediated disorders that may be prevented or treated according to the invention include age-related macular degeneration (AMD) (particularly early (dry) AMD, or geographic atrophy), dense deposit disease (DDD), atypical haemolytic uraemic syndrome (aHUS), C3 glomerulopathies, membranoproliferative glomerulonephritis Type 2 (MPGN2 or MPGN type II), atherosclerosis, chronic cardiovascular disease, Alzheimer's disease, systemic vasculitis, paroxysmal nocturnal haemoglobinuria (PNH), and inflammatory or autoinflammatory diseases of old age.

Examples of complement-mediated disorders that may be prevented or treated according to the invention include: Neurological diseases: such as Guillain Barre Syndrome, AMAN, Miller Fisher Syndrome, Myastenia Gravis, Neuromyelitis Optica, Alzheimer's Disease, or Dry wet AMD; Renal diseases: such as Anti-GBM Nephritis, MPGN I/II, C3G and aHUS, Lupus Nephritis or ANCA Vasculitis; Autoimmune diseases: such as Rheumatoid Arthritis, or Pemphigus Vulgaris/Autoimmune Bullous Dermatoses; transplantation associated pathology: such as Graft versus Host disease (GVHD), ABO-incompatibility of Solid organ, Cellular therapies (HSCT/Car-T) or Instant Blood mediated Inflammatory Reaction (IBMIR); Other diseases include: Paroxysmal nocturnal hemoglobinuria (PNH) or Cardiovascular disease.

Further examples of complement-mediated disorders that may be prevented or treated according to the invention include membranoproliferative glomerulonephritis type I (MPGN type I), membranoproliferative glomerulonephritis type III (MPGN type III), Guillain-Barre syndrome, Henoch-Schönlein purpura, IgA nephropathy, and membranous glomerulonephritis. Membranoproliferative glomerulonephritis (MPGN) is also known as mesangiocapillary glomerulonephritis.

In particular embodiments of the invention, the complement-mediated disorder that is prevented, treated or ameliorated according to the invention is selected from DDD, aHUS, C3 glomerulopathies, atherosclerosis, chronic cardiovascular disease, Alzheimer's disease, systemic vasculitis, PNH, inflammatory or autoinflammatory diseases of old age, MPGN type I, MPGN type III, Guillain-Barre syndrome, Henoch-Schonlein purpura, IgA nephropathy, and membranous glomerulonephritis.

Diseases or disorders that are associated with complement activation are readily identifiable by a person of skill in the art. For example, they may be an autoimmune disease or disorder that involves C1q, such as but not limited to: SLE, rheumatoid arthritis, or cold agglutinin disease.

Other diseases or disorders associated with complement activation involving C1q may be transplantation associated pathology, such as but not limited to hyperacute rejection, chronic rejection, antibody mediated rejection, and ABO-incompatibility, of any of the following: solid organs, bone marrow, hematopoietic stem-cell transplantation and all other cellular products including but not limited to platelets, erythrocytes, leukocytes, stem cells, organoids, and CAR-T cells.

Further diseases or disorders associated with complement activation may be neurological diseases or disorders such as but not limited to Guillain-Barre syndrome (GBS) and Multifocal Motor Neuropathy.

Also, diseases or disorders associated with complement activation may be neurodegenerative diseases or disorders such as but not limited to Alzheimer's disease, amyotrophic lateral sclerosis, multiple sclerosis, glaucoma, myotonic dystrophy, Down syndrome, Parkinson's disease, and Huntington's disease.

In addition, diseases or disorders may comprise the development or progression of cancer. During development or progression of cancer different antigens may be presented by the cancer cells, which may indirectly and/or preferentially recruit C1q to the progressing cancer cell, thereby presenting a target for treatment using the antibodies according to the invention. Diseases or disorders associated with complement activation may include ischemia-reperfusion injury. Ischemia-reperfusion injury activates the complement system via release of damage associated molecular patterns from acutely injured tissue, which then enhances a further immune response.

Diseases or disorders associated with complement activation may also be due to a response of the immune system directed to bacterial infections, myco-bacterial infections, or viral infections.

In a further embodiment the bispecific antibody for use according to the previous aspects of the invention wherein the bispecific antibody is administered systemically or locally, more preferably locally. In another embodiment the bispecific antibody is administered using intra-ocular injection, injection in the joints, skin, or mucosa, or applied as a cream on the skin, eyes or mucosa. The actual amount administered and the rate and time course of administration, will depend on the nature and severity of what is being treated.

The invention further provides a method of treating a subject, comprising administering a therapeutically effective amount of a pharmaceutical composition according to the invention to a subject in need thereof. A therapeutically effective amount refers to an amount sufficient to reduce the severity and/or duration of a disease or a symptom thereof. Progression, development, or onset of the disease may thereby be reduced or prevented. The amount of polypeptide or conjugate actually administered will typically be determined by a physician, in the light of the relevant circumstances, including the condition to be treated, the chosen route of administration, the actual compound administered, the age, weight, and response of the individual patient, the severity of the patient's symptoms, and the like.

In another aspect the present invention provides a bispecific antibody according to the first aspect of the invention conjugated to an additional therapeutic moiety.

In an embodiment the therapeutic moiety is selected from the group consisting of a drug, a toxin, a prodrug, an enzyme, an enzyme that activate a prodrug to a drug, an enzyme-inhibitor, a nuclease, a hormone, a hormone antagonist, an immunomodulator, an oligonucleotide, a boron compound, a photoactive agent or dye, a radionuclide and combinations thereof.

In another aspect the present invention provides a composition comprising the bispecific antibody according to the first aspect of the invention and at least one pharmaceutically acceptable diluent or carrier. In another embodiment the composition includes a complement inhibitor, more preferably the complement inhibitor is selected from the group comprising: a C5a inhibitor, an inhibitor of the receptors for C5a (C5aR1 inhibitor, a/C5aR2) inhibitor, a C3a inhibitor, an inhibitor of the receptor for C3a (C3aR) inhibitor, a factor D inhibitor, a factor B inhibitor, a factor P inhibitor, a FHR-1 inhibitor, a FHR-2 inhibitor, a FHR-3 inhibitor, a FHR-4 inhibitor, a FHR-5 inhibitor,, a C1q inhibitor, a C2 inhibitor, C3 inhibitor, C4 inhibitor, a C5 inhibitor, a C6 inhibitor, a C7 inhibitor, a C8 inhibitor, a C9 inhibitor, a MBL inhibitor, a MASP1 inhibitor, a MASP2 inhibitor, a MASP3 inhibitor, a Ficolin1 inhibitor, a Ficolin2 inhibitor, a Ficolin3 inhibitor, a Collectin10 inhibitor, a Collectin11 inhibitor, an inhibitor of the complement fragments as present in the C3-convertases or C5-convertases such as C3b, C4b, Bb, C2a and the convertases C4b2a, C3bBb, C3bBbP, C4b2aC3b, C3bBbC3b or can be FH, C4BP, C1-INH, complement receptor 1 (CR1, CD35), membrane cofactor protein (MCP, CD46), decay-accelerating factor (DAF, CD55), CD59, FHL-1, mini-FH, factor I (FI), MAp44 (MBL-associated protein 1; MAP-1) and small MBL-associated protein (sMAP; also termed MAp19 or MAP-2), truncated or mini-versions of the complement proteins or any combination thereof.

A composition according to the invention may also include more than one active compound for the disease to be treated. Preferably, the composition comprises an antibody according to the invention and at least one additional active compound that do not adversely affect each other.

The term “antibody”, as used herein, means any antigen-binding molecule that specifically binds to or interacts with a particular antigen (e.g., C4BP). The term “antibody” includes immunoglobulin molecules comprising four polypeptide chains, two heavy (H) chains and two light (L) chains optionally inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3 and potentially additional domains or subunits dependent on the isotype used. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The term antibody as used herein includes antigen-binding molecules exclusively consisting of heavy-chains or light-chains (e.g. heavy chain-only antibodies)

The term “antibody” is intended to refer to an immunoglobulin molecule, a fragment of an immunoglobulin molecule, or a derivative of either thereof, which has the ability to specifically bind to an antigen under typical physiological conditions with a half-life of significant periods of time, such as at least about 30 minutes, at least about 45 minutes, at least about one hour, at least about two hours, at least about four hours, at least about 8 hours, at least about 12 hours, about 24 hours or more, about 48 hours or more, about 3, 4, 5, 6, 7 or more days, etc., or any other relevant functionally-defined period (such as a time sufficient to induce, promote, enhance, and/or modulate a physiological response associated with antibody binding to the antigen and/or time sufficient for the antibody to recruit an effector activity). The antigen-binding region (or antigen-binding domain) which interacts with an antigen may comprise variable regions of both the heavy and light chains of the immunoglobulin molecule or may be a single-domain antigen-binding region, e.g. a heavy chain-variable region only. The constant regions of an antibody, if present, may mediate the binding of the immunoglobulin to host tissues or factors, including various cells of the immune system (such as effector cells and T cells) and components of the complement system such as C1q, the first component in the classical pathway of complement activation.

The Fc region of an immunoglobulin is defined as the fragment of an antibody which would be typically generated after digestion of an antibody with papain which includes the two CH2-CH3 regions of an immunoglobulin and a connecting region, e.g. a hinge region. The constant domain of an antibody heavy chain defines the antibody isotype, e.g. IgG1, IgG2, IgG3, IgG4, IgA1, IgA2, IgM, IgD, or IgE. The Fc-region mediates the effector functions of antibodies with cell surface receptors called Fc receptors and proteins of the complement system. The term “hinge region” as used herein is intended to refer to the hinge region of an immunoglobulin heavy chain. Thus, for example, the hinge region of a human IgG1 antibody corresponds to amino acids 216-230 according to the EU numbering.

The term “CH2 region” or “CH2 domain” as used herein is intended to refer to the CH2 region of an immunoglobulin heavy chain. Thus, for example the CH2 region of a human IgG1 antibody corresponds to amino acids 231-340 according to the EU numbering. However, the CH2 region may also be any of the other subtypes as described herein.

The term “CH3 region” or “CH3 domain” as used herein is intended to refer to the CH3 region of an immunoglobulin heavy chain. Thus, for example the CH3 region of a human IgG1 antibody corresponds to amino acids 341-447 according to the EU numbering. However, the CH3 region may also be any of the other subtypes as described herein.

Reference to amino acid positions in the constant domain in the present invention is according to the EU-numbering (Edelman et al., Proc Natl Acad Sci USA. 1969 May;63 (1): 78-85; Kabat et al., Sequences of proteins of immunological interest. 5th Edition-1991 NIH Publication No. 91-3242). For the avoidance of doubt for variable domains, the numbering used herein is IMGT.

As indicated above, the term antibody as used herein, unless otherwise stated or clearly contradicted by context, includes fragments of an antibody that retain the ability to specifically bind to the antigen. It has been shown that the antigen-binding function of an antibody may be performed by fragments of a full-length antibody. Examples of binding fragments encompassed within the term “antibody” include (i) a Fab′ or Fab fragment, i.e. a monovalent fragment consisting of the VL, VH, CL and CH1 domains, or a monovalent antibody as described in WO2007059782; (ii) F(ab′) 2 fragments, i.e. bivalent fragments comprising two Fab fragments linked by a disulfide bridge at the hinge region; (iii) a Fd fragment consisting essentially of the VH and CH1 domains; and (iv) a Fv fragment consisting essentially of the VL and VH domains of a single arm of an antibody. Furthermore, although the two domains of the Fv fragment, VL and VH, are coded for by separate genes, they may be joined, using recombinant methods, by a synthetic linker that enables them to be made as a single protein chain in which the VL and VH regions pair to form monovalent molecules (known as single chain antibodies or single chain Fv (scFv), see for instance Bird et al., Science 242, 423-426 (1988) and Huston et al., PNAS USA 85, 5879-5883 (1988)). Such single chain antibodies are encompassed within the term antibody unless otherwise indicated by context. Although such fragments are generally included within the meaning of antibody, they collectively and each independently are unique features of the present invention, exhibiting different biological properties and utility. The term antibody, unless specified otherwise, also includes polyclonal antibodies, monoclonal antibodies (mAbs), chimeric antibodies and humanized antibodies, and antibody fragments provided by any known technique, such as enzymatic cleavage, peptide synthesis, and recombinant techniques.

In some embodiments of the antibodies of the invention, the first antigen-binding region or the antigen-binding region, or both, is a single domain antibody. Single domain antibodies (sdAb, also called Nanobody®, or VHH) are well known to the skilled person, see e.g. Hamers-Casterman et al. (1993) Nature 363:446, Roovers et al. (2007) Curr Opin Mol Ther 9:327 and Krah et al. (2016) Immunopharmacol Immunotoxicol 38:21. Single domain antibodies comprise a single CDR1, a single CDR2 and a single CDR3. Examples of single domain antibodies are variable fragments of heavy chain-only antibodies, antibodies that naturally do not comprise light chains, single domain antibodies derived from conventional antibodies, and engineered antibodies. Single domain antibodies may be derived from any species including mouse, human, camel, llama, shark, goat, rabbit, and cow. For example, naturally occurring VHH molecules can be derived from antibodies raised in Camelidae species, for example in camel, dromedary, llama, alpaca and guanaco. Like a whole antibody, a single domain antibody is able to bind selectively to a specific antigen. Single domain antibodies may contain only the variable domain of an immunoglobulin chain, i.e. CDR1, CDR2 and CDR3 and framework regions. The term “immunoglobulin” as used herein is intended to refer to a class of structurally related glycoproteins consisting of two pairs of polypeptide chains, one pair of light (L) chains and one pair of heavy (H) chains, all four potentially inter-connected by disulfide bonds. The term “immunoglobulin heavy chain”, “heavy chain of an immunoglobulin” or “heavy chain” as used herein is intended to refer to one of the chains of an immunoglobulin. A heavy chain is typically comprised of a heavy chain variable region (abbreviated herein as VH) and a heavy chain constant region (abbreviated herein as CH) which defines the isotype of the immunoglobulin. The heavy chain constant region typically is comprised of three domains, CH1, CH2, and CH3. The heavy chain constant region further comprises a hinge region. Within the structure of the immunoglobulin (e.g. lgG), the two heavy chains are inter-connected via disulfide bonds in the hinge region. Equally to the heavy chains, each light chain is typically comprised of several regions; a light chain variable region (VL) and a light chain constant region (CL). Furthermore, the VH and VL regions may be subdivided into regions of hypervariability (or hypervariable regions which may be hypervariable in sequence and/or form structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4. CDR sequences may be determined by use of various methods, e.g. the methods provided by Chothia and Lesk (1987) J. Mol. Biol. 196:901 or Kabat et al. (1991) Sequence of protein of immunological interest, fifth edition. NIH publication. Various methods for CDR determination and amino acid numbering can be compared on www.abysis.org (UCL).

The term “isotype” as used herein, refers to the immunoglobulin (sub) class (for instance IgG1, IgG2, IgG3, IgG4, IgD, IgA, IgE, or IgM) or any allotype thereof, such as IgG1m(za) and IgG1m(f) that is encoded by heavy chain constant region genes. Each heavy chain isotype can be combined with either a kappa (κ) or lambda (λ) light chain. An antibody of the invention can possess any isotype.

The term “parent antibody”, is to be understood as an antibody which is identical to an antibody according to the invention, but wherein the parent antibody does not have one or more of the specified mutations. A “variant” or “antibody variant” or a “variant of a parent antibody” of the present invention is an antibody molecule which comprises one or more mutations as compared to a “parent antibody”. Amino acid substitutions may exchange a native amino acid for another naturally-occurring amino acid, or for a non-naturally-occurring amino acid derivative. The amino acid substitution may be conservative or non-conservative. In the context of the present invention, conservative substitutions may be defined by substitutions within the classes of amino acids reflected in one or more of the following three tables:

### Amino Acid Residue Classes for Conservative Substitutions

| | |
|---|---|
| Acidic Residues | Asp (D) and Glu (E) |
| Basic Residues | Lys (K), Arg (R), and His (H) |
| Hydrophilic Uncharged | Ser (S), Thr (T), Asn (N), and |
| Residues | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

### Alternative Conservative Amino Acid Residue Substitution Classes

| | | | |
|---|---|---|---|
| 1 | A | S | T |
| 2 | D | E | |
| 3 | N | Q | |
| 4 | R | K | |
| 5 | I | L | M |
| 6 | F | Y | W |

### Alternative Physical and Functional Classifications of Amino Acid Residues

| | |
|---|---|
| Alcohol group-containing | S and T |
| residues | |
| Aliphatic residues | I, L, V, and M |
| Cycloalkenyl-associated | F, H, W, and Y |
| residues | |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn | A, C, D, E, G, H, K, N, Q, R, S, |
| formation | P, and T |
| Flexible residues | Q, T, K, S, G, N, D, E, and R |

In the context of the present invention, a substitution in a variant is indicated as:
Original amino acid—position—substituted amino acid;

The three-letter code, or one letter code, are used, including the codes Xaa and X to indicate amino acid residue. Accordingly, the notation “T366W” means that the variant comprises a substitution of threonine with tryptophan in the variant amino acid position corresponding to the amino acid in position 366 in the parent antibody.

Furthermore, the term “a substitution” embraces a substitution into any one of the other nineteen natural amino acids, or into other amino acids, such as non-natural amino acids.

The term “full-length antibody” when used herein, refers to an antibody which contains all heavy and light chain constant and variable domains corresponding to those that are normally found in a wild-type antibody of that isotype.

The term “chimeric antibody” refers to an antibody wherein the variable region is derived from a non-human species (e.g. derived from rodents) and the constant region is derived from a different species, such as human. Chimeric antibodies may be generated by genetic engineering. Chimeric monoclonal antibodies for therapeutic applications are developed to reduce antibody immunogenicity.

The term “humanized antibody” refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), which together form the antigen binding site, onto a homologous human acceptor framework region (FR). In order to fully reconstitute the binding affinity and specificity of the parental antibody, the substitution of framework residues from the parental antibody (i.e. the non-human antibody) into the human framework regions (back-mutations) may be required. Structural homology modelling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and, optionally, fully human constant regions. Optionally, additional amino acid modifications, which are not necessarily back-mutations, may be introduced to obtain a humanized antibody with preferred characteristics, such as affinity and biochemical properties. Humanization of non-human therapeutic antibodies is performed to minimize its immunogenicity in man while such humanized antibodies at the same time maintain the specificity and binding affinity of the antibody of non-human origin.

The term “multispecific antibody” refers to an antibody having specificities for at least two different, such as at least three, typically non-overlapping, epitopes. Such epitopes may be on the same or on different target antigens. If the epitopes are on different targets, such targets may be on the same cell or different cells or cell types. In some embodiments, a multispecific antibody may comprise one or more single-domain antibodies.

The term “bispecific antibody” refers to an antibody having specificities for two different, typically non-overlapping, epitopes. Such epitopes may be on the same or different targets. If the epitopes are on different targets, such targets may be on the same cell or different cells or cell types. In some embodiments, a bispecific antibody may comprise one or two single-domain antibodies.

Examples of different classes of multispecific, such as bispecific, antibodies include but are not limited to (i) IgG-like molecules with complementary CH3 domains to force heterodimerization; (ii) recombinant IgG-like dual targeting molecules, wherein the two sides of the molecule each contain the Fab fragment or part of the Fab fragment of at least two different antibodies; (iii) IgG fusion molecules, wherein full length IgG antibodies are fused to extra Fab fragment or parts of Fab fragment; (iv) Fc fusion molecules, wherein single chain Fv molecules or stabilized diabodies are fused to heavy-chain constant-domains, Fc-regions or parts thereof; (v) Fab fusion molecules, wherein different Fab-fragments are fused together, fused to heavy-chain constant-domains, Fc-regions or parts thereof; and (vi) ScFv-and diabody-based and heavy chain antibodies (e.g., domain antibodies, Nanobodies®) wherein different single chain Fv molecules or different diabodies or different heavy-chain antibodies (e.g. domain antibodies, Nanobodies®) are fused to each other or to another protein or carrier molecule fused to heavy-chain constant-domains, Fc-regions or parts thereof.

Examples of IgG-like molecules with complementary CH3 domains molecules include but are not limited to the Triomab® (Trion Pharma/Fresenius Biotech), the Knobs-into-Holes (Genentech), CrossMAbs (Roche) and the electrostatically-matched (Amgen, Chugai, Oncomed), the LUZ-Y (Genentech, Wranik et al. J. Biol. Chem. 2012, 287 (52): 43331-9, doi: 10.1074/jbc.M112.397869. Epub 2012 Nov. 1), DIG-body and PIG-body (Pharmabcine, WO2010134666, WO2014081202), the Strand Exchange Engineered Domain body (SEEDbody) (EMD Serono), the Biclonics (Merus, WO2013157953), FcAAdp (Regeneron), bispecific IgG1 and IgG2 (Pfizer/Rinat), Azymetric scaffold (Zymeworks/Merck,), mAb-Fv (Xencor), bivalent bispecific antibodies (Roche, WO2009080254) and DuoBody® molecules (Genmab).

Examples of recombinant IgG-like dual targeting molecules include but are not limited to Dual Targeting (DT)-Ig (GSK/Domantis, WO2009058383), Two-in-one Antibody (Genentech, Bostrom, et al 2009. Science 323, 1610-1614), Cross-linked Mabs (Karmanos Cancer Center), mAb2 (F-Star), Zybodies™ (Zyngenia, LaFleur et al. MAbs. 2013 March-Apr;5 (2): 208-18), approaches with common light chain, κλBodies (Novimmune, WO2012023053) and CovX-body® (CovX/Pfizer, Doppalapudi, V. R., et al 2007. Bioorg. Med. Chem. Lett. 17,501-506).

Examples of IgG fusion molecules include but are not limited to Dual Variable Domain (DVD)-Ig (Abbott), Dual domain double head antibodies (Unilever; Sanofi Aventis), IgG-like Bispecific (ImClone/Eli Lilly, Lewis et al. Nat Biotechnol. 2014 February;32 (2): 191-8), Ts2Ab (MedImmune/AZ, Dimasi et al. J Mol Biol. 2009 Oct. 30;393 (3): 672-92) and BsAb (Zymogenetics, WO2010111625), HERCULES (Biogen Idec), scFv fusion (Novartis), scFv fusion (Changzhou Adam Biotech Inc) and TvAb (Roche).

Examples of Fc fusion molecules include but are not limited to ScFv/Fc Fusions (Academic Institution, Pearce et al Biochem Mol Biol Int. 1997 September;42 (6): 1179), SCORPION (Emergent BioSolutions/Trubion, Blankenship J W, et al. AACR 100th Annual meeting 2009 (Abstract #5465); Zymogenetics/BMS, WO2010111625), Dual Affinity Retargeting Technology (Fc-DART™) (MacroGenics) and Dual (ScFv) 2-Fab (National Research Center for Antibody Medicine—China).

Examples of Fab fusion bispecific antibodies include but are not limited to F (ab) 2 (Medarex/AMGEN), Dual-Action or Bis-Fab (Genentech), Dock-and-Lock® (DNL) (ImmunoMedics), Bivalent Bispecific (Biotecnoland Fab-Fv (UCB-Celltech).

Examples of ScFv-, diabody-based and domain antibodies include but are not limited to Bispecific T Cell Engager (BiTER) (Micromet, Tandem Diabody (Tandab) (Affimed), Dual Affinity Retargeting Technology (DART™) (MacroGenics), Single-chain Diabody (Academic, Lawrence FEBS Lett. 1998 Apr. 3;425 (3): 479-84), TCR-like Antibodies (AIT, ReceptorLogics), Human Serum Albumin ScFv Fusion (Merrimack, WO2010059315) and COMBODY molecules (Epigen Biotech, Zhu et al. Immunol Cell Biol. 2010 August;88 (6): 667-75), dual targeting Nanobodies® (Ablynx, Hmila et al., FASEB J. 2010), dual targeting heavy chain only domain antibodies.

In the context of antibody binding to an antigen, the terms “binds” or “specifically binds” refer to the binding of an antibody to a predetermined antigen or target to which binding typically is with an apparent affinity corresponding to a K_{D} of about 10⁻⁶ M or less, e.g. 10⁻⁷ M or less, such as about 10⁻⁸ M or less, such as about 10⁻⁹ M or less, about 10⁻¹⁰ M or less, or about 10⁻¹¹ M or even less, e.g. when determined using flow cytometry as described in the Examples herein. Alternatively, K_{D} values can be determined using for instance surface plasmon resonance (SPR) technology in a BIAcore T200 or bio-layer interferometry (BLI) in an Octet RED96 instrument using the antigen as the ligand and the binding moiety or binding molecule as the analyte. Specific binding means that the antibody binds to the predetermined antigen with an affinity corresponding to a K_{D} that is at least ten-fold lower, such as at least 100-fold lower, for instance at least 1,000 fold lower, such as at least 10,000 fold lower, for instance at least 100,000 fold lower than its affinity for binding to a non-specific antigen (e.g., BSA, casein) other than the predetermined antigen or a closely-related antigen. The degree with which the affinity is lower is dependent on the K_{D} of the binding moiety or binding molecule, so that when the K_{D} of the binding moiety or binding molecule is very low (that is, the binding moiety or binding molecule is highly specific), then the degree with which the affinity for the antigen is lower than the affinity for a non-specific antigen may be at least 10,000-fold. The term “K_{D}” (M), as used herein, refers to the dissociation equilibrium constant of a particular interaction between the antigen and the binding moiety or binding molecule. Methods for measuring binding specificity are well known in the art. For example, as described in the experimental section herein.

The terms “antigen-binding portion” of an antibody, “antigen-binding fragment” of an antibody, “antigen binding domain” of an antibody and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (I) Fab fragments; (ii) F(ab′)₂ fragments; (iii) Fd fragments; (iv) Fv fragments; (v) single-chain Fv (scFv) molecules; (vi) dAb fragments; and (vil) minimal recognition units consisting of the amino acid residues that mimic the hypervariable region of an antibody (e.g., an isolated complementarity determining region (CDR). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

In certain embodiments of the invention, the bispecific antibodies of the invention are human antibodies. The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The antibodies of the invention may, in some embodiments, be recombinant human antibodies. The term “recombinant human antibody”, as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The antibodies of the present invention can be derived from a mouse, human or other species. The antibodies of the invention may, in some embodiments, be recombinant mouse, rat, lama or rabbit. The antibodies of the invention may be humanized antibodies or chimeric antibodies.

The antibodies of the invention may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The bispecific antibodies disclosed herein may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). The bispecific antibodies disclosed herein may comprise additional mutations i.e. naturally occurring mutations, compared to the original antibody sequence. Human antibodies may, in certain embodiments, include mutations introduced by random or site-specific mutagenesis in vitro or by somatic mutation in vivo.

As used herein, the expression “bispecific antigen-binding molecule” or “bispecific antibody” means a protein, polypeptide or molecular complex comprising at least a first antigen-binding domain and a second antigen-binding domain. Each antigen-binding domain within the bispecific antigen-binding molecule or bispecific antibody comprises at least one CDR that alone, or in combination with one or more additional CDRs and/or FRs, specifically binds to a particular antigen. In the context of the present invention, the first antigen-binding domain specifically binds a first antigen (e.g., C4BP, or FH), and the second antigen-binding domain specifically binds a second, distinct antigen (e.g., tissue specific antigens, autoantigens, such as the glomerular basement membrane, collagen, the acetylcholine receptor, Human Leucocyte Antigens or Tissue Factor), The term “autoantigen” as used herein refers to a normal protein or complex of proteins that is recognized by the immune system of patients suffering from a specific autoimmune disease. The present invention provides a form of personalised medicine by targeting complement inhibitors to the sites where autoantibodies are triggering complement activation and thereby treat autoimmune diseases or conditions.

In certain exemplary embodiments of the present invention, the bispecific antigen-binding molecule is a bispecific antibody. Each antigen-binding domain of a bispecific antibody comprises a heavy chain variable domain (HCVR) and a light chain variable domain (LCVR). The first antigen-binding domain and the second antigen-binding domain may be directly or indirectly connected to one another to form a bispecific antigen-binding molecule of the present invention. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin C_{H}3 domain. A non-limiting example of a multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

In certain embodiments, the multimerizing domains include peptides or polypeptides comprising or consisting of a leucine zipper, a helix-loop motif, or a coiled-coil motif.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule.

Exemplary bispecific antibodies of the present invention are listed in Table 2 and Table 3 herein. Table 2 sets forth the amino acid sequence identifiers of the heavy chain variable regions (HCVRs) and light chain variable regions (LCVRs), Table 3 sets forth the heavy chain complementarity determining regions (HCDR1, HCDR2 and HCDR3), and light chain complementarity determining regions (LCDR1, LCDR2 and LCDR3) of the exemplary bispecific antibodies.

In another aspect of the present invention the bispecific antibodies according to the first aspect are typically produced recombinantly, i.e. by expression of nucleic acid constructs encoding the antibodies in suitable host cells, followed by purification of the produced recombinant antibody from the cell culture. Nucleic acid constructs can be produced by standard molecular biological techniques well-known in the art. The constructs are typically introduced into the host cell using an expression vector. Suitable nucleic acid constructs and expression vectors are known in the art. Host cells suitable for the recombinant expression of antibodies are well-known in the art, and include CHO, HEK-293, Expi293F, PER-C6, NS/0 and Sp2/0 cells. Accordingly, in a further aspect, the invention relates to a nucleic acid construct encoding a bispecific antibody according to the first aspect of the invention. In one embodiment, the construct is a DNA construct. In another embodiment, the construct is an RNA construct. In a further aspect, the invention relates to an expression vector comprising a nucleic acid construct encoding a bispecific antibody according to the first aspect of the invention. In a further aspect, the invention relates to a host cell comprising one or more nucleic acid constructs encoding a bispecific antibody according to the first aspect of the invention or an expression vector comprising a nucleic acid construct encoding a bispecific antibody according to the first aspect of the invention.

In a further aspect, the invention relates to a process for manufacturing the bispecific antibody according to the first aspect of the invention, comprising expressing one or more nucleic acids encoding the bispecific antibody according to the first aspect of the invention in a host cell. In a further aspect, the invention the relates to a process for manufacturing a clinical batch of the bispecific antibody according to a first aspect of the invention, comprising expressing one or more nucleic acids encoding the bispecific antibody according to the first aspect of the invention in a host cell. A “clinical batch” when used herein refers to a product composition that is suitable for use in humans.

An aspect of the invention provides a method of making a bispecific antibody comprising a chimeric constant heavy chain region, said method comprising: (a) transfecting a host cell with a nucleic acid molecule encoding a first light chain capable of binding the complement inhibitor antigen, said nucleic acid molecule comprising a nucleotide sequence encoding the VL region of the first and a nucleotide sequence encoding the constant CL region of an Ig, wherein said nucleotide sequence encoding the VL region of a selected antigen-specific antibody and said nucleotide sequence encoding the CL region of an Ig are operably linked together; (b) transfecting the host cell of step (a) with a nucleic acid molecule encoding a first heavy chain of the antibody capable of binding the complement inhibitor antigen, said nucleic acid molecule comprising a nucleotide sequence encoding the VH region and a nucleotide sequence encoding a chimeric constant CH region of a human Ig, wherein said nucleotide sequence encoding the VH region and the nucleotide sequence encoding the CH region of said Ig are operably linked together; (c) transfecting the host cell of step (a) with a nucleic acid molecule encoding a second heavy chain of the antibody capable of binding the target antigen, said nucleic acid molecule comprising a nucleotide sequence encoding the VH region and a nucleotide sequence encoding a chimeric CH region of a human Ig, wherein said nucleotide sequence encoding the VH region and the nucleotide sequence encoding the CH region of said Ig are operably linked together; and (c) making said antibody by co-expressing the nucleic acid molecules of (a) and (b) in said host cell.

In some aspects, the method of making the bispecific antibody optionally comprises transfecting the host cell of step (a) with a nucleic acid molecule encoding a second light chain capable of binding the target antigen, said nucleic acid molecule comprising a nucleotide sequence encoding the VL region of the second light chain and a nucleotide sequence encoding the constant CL region of an Ig, wherein said nucleotide sequence encoding the VL region of the second light chain and said nucleotide sequence encoding the CL region of an Ig are operably linked together

In another aspect of the present invention, a method of making the bispecific antibody of the present invention is performed using the Controlled Fab-arm exchange.

Thus, the present invention includes bispecific antibodies wherein one arm of an immunoglobulin binds a complement inhibitor, and the other arm of the immunoglobulin is specific for a target antigen. The target antigen that the other arm of the complement inhibitor bispecific antibody binds can be any antigen expressed on or in the vicinity of a cell, tissue, organ, microorganism or virus, against which a targeted immune response is desired. The complement inhibitor binding arm can comprise any of the HCVR/LCVR or CDR amino acid sequences SEQ ID NOs: 15, 16, 17, 18, 19, and 20 or 21, 22, 23, 24, 25 and 26, as set forth in Table 1, 2 and 3 herein.

Additional advantages of using endogenous complement inhibitors include the non-immunogenic nature as well as the fact that endogenous inhibitors like FH and C4BP can actually perform their inhibitory function multiple times, both regarding cofactor activity towards FI as well as the decay-accelerating activity. This is in sharp contrast to immunogenic constructs that artificially combine domains from different molecules or the complement-depleting nature of the currently used anti-complement biologicals.

The C4BP-targeting bsAbs do not inhibit activation of the alternative pathway, which was to be expected since the alternative pathway does not involve C4b. Therefore, depending on whether the disease is more classical or lectin pathway-mediated or alternative pathway-mediated, either C4BP or FH could be targeted, respectively.

The other arm can be designed to bind specific (auto) antigens, for example collagen in arthritis (1), targeting the regulator to the joints, locally inhibiting complement activation. In nephritis, the glomerular basement membrane could be targeted (2), bringing the regulator to the kidneys, and in myasthenia gravis, the acetylcholine receptor can be targeted (3), causing the regulator to go to the neuromuscular junction. A final example is transplant rejection, in which a transplanted organ that is not fully HLA-matched elicits an immune response (4). In this case, the transplanted organ could be coated with our bsAb binding to FH or C4BP and HLA, shielding the antigenic epitopes, but also targeting a complement regulator to the transplanted organ, protecting the organ from complement-mediated attack.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Preferred features and embodiments of the invention are as for the other aspects mutatis mutandis.

Aspects of the invention are demonstrated by the following non-limiting examples.

### EXAMPLES

Controlled Fab-arm exchange was used to generate a comprehensive set of bi-specific antibodies (bsAbs) that with one arm bind to a site-specific target and with the other arm bind to the endogenous complement regulators such as factor H (FH) or C4b-binding protein (C4BP). The engineered bsAbs and their controls were tested in several complement assays, monitoring complement activation and lysis.

It was observed that the engineered bsAbs were able to inhibit complement activation and lysis. In plate-bound assays the bsAbs were capable of inhibiting complement activation initiated by the classical, lectin, and alternative pathway. The concentrations of endogenous FH and C4BP were sufficient to mediate local inhibition. The bsAbs were also able to protect model-liposomes from complement-mediated lysis as well as erythrocytes in classical and alternative pathway assays. Finally, the bsAbs were capable of protecting human leukocytes from complement-mediated lysis.

In conclusion, the novel format of bsAbs binding specific (auto) antigens as well as FH or C4BP is able to locally inhibit complement using human endogenous complement inhibitors, providing a novel therapeutic approach for the treatment of complement-mediated diseases.

### Example 1. Generation of Bispecific Antibodies

A comprehensive set of bispecific antibodies (bsAbs) were generated to target complement inhibitors factor H (FH) or C4b-binding protein (C4BP) to specific locations.

### Antibody Sequences

The variable heavy (VH) and variable light (VL) sequences of anti-FH (OX-24) and anti-C4BP (3B9D6) were obtained by sequencing hybridomas (see below). Anti-biotin (8), anti-CD20 (rituximab, DrugBank Accession Number DB00073), anti-CD52 (alemtuzumab, (9)), anti-DNP (10), anti-HIV (b12, (11)), and anti-HLA I (W6-32, (12)) VH and VL sequences were extracted from literature or an online database and are outlined in table 1 below.

Table 1: Amino acid sequences for parental antibodies anti-FH, anti-C4BP, anti-DNP, anti-DNP, anti-HIV, anti-biotin and anti-HLA I. The variable domains are underlined. The grey highlighted amino acid are the mutations that are used to perform the Fab-arm exchange.

| Parental | | |
|---|---|---|
| Antibody | Heavy chain | Light chain |
| Anti-FH | EVQLQQSGPELVKPGASVKISC | DILMTQSPSSMSVSLGDTVSITCHA |
| F405L | KASGYSFTGYFIHWVKQSHVKS | SQGITSNIGWLQQKPGKSFKGLIYH |
| LALAPG | LEWFGRINPYNGATDY | GTNLEDGVPS |
| | NQNFKDKAILTIDESSSTAYMEIH | RFSGSGSGADYSLTISSLESEDFA |
| | SLTSDDSAVYYCARGLFLDYWG | DYYCVQYAQFPWTFGGGTKLEIKR |
| | QGTTLTVSSASTKG | TVAAPSVFIFPP |
| | PSVFPLAPSSKSTSGGTAALGCL | SDEQLKSGTASVVCLLNNFYPREA |
| | VKDYFPEPVTVSWNSGALTSGV | KVQWKVDNALQSGNSQESVTEQD |
| | HTFPAVLQSSGLYSL | SKDSTYSLSSTLT |
| | SSVVTVPSSSLGTQTYICNVNHK | LSKADYEKHKVYACEVTHQGLSSP |
| | PSNTKVDKRVEPKSCDKTHTCP | VTKSFNRGEC* (SEQ ID NO: 2) |
| | PCPAPEAAGGPSVFL | |
| | FPPKPKDTLMISRTPEVTCVVVD | |
| | VSHEDPEVKFNWYVDGVEVHN | |
| | AKTKPREEQYNSTYRV | |
| | VSVLTVLHQDWLNGKEYKCKVS | |
| | NKALGAPIEKTISKAKGQPREPQ | |
| | VYTLPPSREEMTKNQ | |
| | VSLTCLVKGFYPSDIAVEWESNG | |
| | QPENNYKTTPPVLDSDGSF [symbol] LYS | |
| | KLTVDKSRWQQGNV | |
| | FSCSVMHEALHNHYTQKSLSLS | |
| | PGK* (SEQ ID NO: 1) | |
| Anti-C4BP | EVQLVESGGGLVKPGGSLKLSC | DVVMTQTPLTLSITIGQPASISCKSS |
| F405L | AVSGFIFSDYYMYWVRQTPEKR | QSLLDGDGKTYLNWLLQRPGQSP |
| LALAPG | LEWVATISDGGVYTYY | KRLIYLVSKLD |
| | PDSMKGRFTISRDNAKNNLYLQ | SGVPDRFTGSGSGTDFTLKISRVE |
| | MSSLKSEDTAMYYCVRGDYYG | AEDLGVYYCCQGTHFPWTFGRGT |
| | NSLDYWGQGTSVTVSSA | KLEIKRTVAAPSV |
| | STKGPSVFPLAPSSKSTSGGTA | FIFPPSDEQLKSGTASVVCLLNNFY |
| | ALGCLVKDYFPEPVTVSWNSGA | PREAKVQWKVDNALQSGNSQESV |
| | LTSGVHTFPAVLQSSG | TEQDSKDSTYSL |
| | LYSLSSVVTVPSSSLGTQTYICN | SSTLTLSKADYEKHKVYACEVTHQ |
| | VNHKPSNTKVDKRVEPKSCDKT | GLSSPVTKSFNRGEC* (SEQ ID NO: |
| | HTCPPCPAPEAAGGP | 4) |
| | SVFLFPPKPKDTLMISRTPEVTC | |
| | VVVDVSHEDPEVKFNWYVDGVE | |
| | VHNAKTKPREEQYNS | |
| | TYRVVSVLTVLHQDWLNGKEYK | |
| | CKVSNKALGAPIEKTISKAKGQP | |
| | REPQVYTLPPSREEM | |
| | TKNQVSLTCLVKGFYPSDIAVEW | |
| | ESNGQPENNYKTTPPVLDSDGS | |
| | F [symbol] LYSKLTVDKSRWQ | |
| | QGNVFSCSVMHEALHNHYTQKS | |
| | LSLSPGK* (SEQ ID NO: 3) | |
| Anti-DNP | DVRLQESGPGLVKPSQSLSLTC | DIRMTQTTSSLSASLGDRVTISCRA |
| K409R | SVTGYSITNSYYWNWIRQFPGN | SQDISNYLNWYQQKPDGTVKLLIY |
| LALAPG | KLEWMVYIGYDGSNNY | YTSRLHSGVPS |
| | NPSLKNRISITRDTSKNQFFLKLN | RFSGSGSGTDYSLTISNLEQEDIAT |
| | SVTTEDTATYYCARATYYGNYR | YFCQQGNTLPWTFGGGTKLEIKRT |
| | GFAYWGQGTLVTVSA | VAAPSVFIFPP |
| | ASTKGPSVFPLAPSSKSTSGG | SDEQLKSGTASVVCLLNNFYPREA |
| | TAALGCLVKDYFPEPVTVSWNS | KVQWKVDNALQSGNSQESVTEQD |
| | GALTSGVHTFPAVLQS | SKDSTYSLSSTLT |
| | SGLYSLSSVVTVPSSSLGTQTYI | LSKADYEKHKVYACEVTHQGLSSP |
| | CNVNHKPSNTKVDKRVEPKSCD | VTKSFNRGEC* (SEQ ID NO: 6) |
| | KTHTCPPCPAPEAAG | |
| | GPSVFLFPPKPKDTLMISRTPEV | |
| | TCVVVDVSHEDPEVKFNWYVDG | |
| | VEVHNAKTKPREEQY | |
| | NSTYRVVSVLTVLHQDWLNGKE | |
| | YKCKVSNKALGAPIEKTISKAKG | |
| | QPREPQVYTLPPSRE | |
| | EMTKNQVSLTCLVKGFYPSDIAV | |
| | EWESNGQPENNYKTTPPVLDSD | |
| | GSFFLYS [symbol] LTVDKSR | |
| | WQQGNVFSCSVMHEALHNHYT | |
| | QKSLSLSPGK* (SEQ ID NO: 5) | |
| Anti-HIV | QVQLVQSGAEVKKPGASVKVSC | EIVLTQSPGTLSLSPGERATFSCRS |
| F405L | QASGYRFSNFVIHWVRQAPGQR | SHSIRSRRVAWYQHKPGQAPRLVI |
| LALAPG | FEWMGWINPYNGNKEF | HGVSNRASGIS |
| | SAKFQDRVTFTADTSANTAYME | DRFSGSGSGTDFTLTITRVEPEDFA |
| | LRSLRSADTAVYYCARVGPYSW | LYYCQVYGASSYTFGQGTKLERKR |
| | DDSPQDNYYMDVWGKG | TVAAPSVFIFP |
| | TTVIVSSASTKGPSVFPLAPSSK | PSDEQLKSGTASVVCLLNNFYPRE |
| | STSGGTAALGCLVKDYFPEPVT | AKVQWKVDNALQSGNSQESVTEQ |
| | VSWNSGALTSGVHTF | DSKDSTYSLSSTL |
| | PAVLQSSGLYSLSSVVTVPSSSL | TLSKADYEKHKVYACEVTHQGLSS |
| | GTQTYICNVNHKPSNTKVDKRV | PVTKSFNRGEC* (SEQ ID NO: 8) |
| | EPKSCDKTHTCPPCP | |
| | APEAAGGPSVFLFPPKPKDTLMI | |
| | SRTPEVTCVVVDVSHEDPEVKF | |
| | NWYVDGVEVHNAKTK | |
| | PREEQYNSTYRVVSVLTVLHQD | |
| | WLNGKEYKCKVSNKALGAPIEK | |
| | TISKAKGQPREPQVYT | |
| | LPPSREEMTKNQVSLTCLVKGF | |
| | YPSDIAVEWESNGQPENNYKTT | |
| | PPVLDSDGSF [symbol] LYSKL | |
| | TVDKSRWQQGNVFSCSVMHEA | |
| | LHNHYTQKSLSLSPGK* (SEQ ID | |
| | NO: 7) | |
| Anti-HIV | QVQLVQSGAEVKKPGASVKVSC | EIVLTQSPGTLSLSPGERATFSCRS |
| K409R | QASGYRFSNFVIHWVRQAPGQR | SHSIRSRRVAWYQHKPGQAPRLVI |
| LALAPG | FEWMGWINPYNGNKEF | HGVSNRASGIS |
| | SAKFQDRVTFTADTSANTAYME | DRFSGSGSGTDFTLTITRVEPEDFA |
| | LRSLRSADTAVYYCARVGPYSW | LYYCQVYGASSYTFGQGTKLERKR |
| | DDSPQDNYYMDVWGKG | TVAAPSVFIFP |
| | TTVIVSSASTKGPSVFPLAPSSK | PSDEQLKSGTASVVCLLNNFYPRE |
| | STSGGTAALGCLVKDYFPEPVT | AKVQWKVDNALQSGNSQESVTEQ |
| | VSWNSGALTSGVHTF | DSKDSTYSLSSTL |
| | PAVLQSSGLYSLSSVVTVPSSSL | TLSKADYEKHKVYACEVTHQGLSS |
| | GTQTYICNVNHKPSNTKVDKRV | PVTKSFNRGEC* SEQ ID NO: 10) |
| | EPKSCDKTHTCPPCP | |
| | APEAAGGPSVFLFPPKPKDTLMI | |
| | SRTPEVTCVVVDVSHEDPEVKF | |
| | NWYVDGVEVHNAKTK | |
| | PREEQYNSTYRVVSVLTVLHQD | |
| | WLNGKEYKCKVSNKALGAPIEK | |
| | TISKAKGQPREPQVYT | |
| | LPPSREEMTKNQVSLTCLVKGF | |
| | YPSDIAVEWESNGQPENNYKTT | |
| | PPVLDSDGSFFLYS [symbol] L | |
| | TVDKSRWQQGNVFSCSVMHEA | |
| | LHNHYTQKSLSLSPGK* (SEQ ID | |
| | NO: 9) | |
| Anti-biotin | QVKLQESGPGLVAPSQSLSITCT | DVVMTQSPLSLPVSLGDQASISCR |
| K409R | VSGFSLTAYGVDWVRQPPGKGL | SGQGLVYSNGDTYLHWYLQKPGQ |
| LALAPG | EWLGVIWGGGRTNYN | SPKLLIYKVSNRE |
| | SGLMSRLSIRKDNSKSQVFLTM | SGVPDRFSGSGSGTDFTLKISRVE |
| | NSLQTDDTAKYYCVKHTNWDG | AEDLGVYFCSQSTHFPFTFGSGTK |
| | GFAYWGQGTTVTVSSAS | LEIKRTVAAPSV |
| | TKGPSVFPLAPSSKSTSGGTAAL | FIFPPSDEQLKSGTASVVCLLNNFY |
| | GCLVKDYFPEPVTVSWNSGALT | PREAKVQWKVDNALQSGNSQESV |
| | SGVHTFPAVLQSSGL | TEQDSKDSTYSL |
| | YSLSSVVTVPSSSLGTQTYICNV | SSTLTLSKADYEKHKVYACEVTHQ |
| | NHKPSNTKVDKRVEPKSCDKTH | GLSSPVTKSFNRGEC* (SEQ ID NO: |
| | TCPPCPAPEAAGGPS | 12) |
| | VFLFPPKPKDTLMISRTPEVTCV | |
| | VVDVSHEDPEVKFNWYVDGVEV | |
| | HNAKTKPREEQYNST | |
| | YRVVSVLTVLHQDWLNGKEYKC | |
| | KVSNKALGAPIEKTISKAKGQPR | |
| | EPQVYTLPPSREEMT | |
| | KNQVSLTCLVKGFYPSDIAVEWE | |
| | SNGQPENNYKTTPPVLDSDGSF | |
| | FLYS [symbol] LTVDKSRWQQ | |
| | GNVFSCSVMHEALHNHYTQKSL | |
| | SLSPGK* (SEQ ID NO 11) | |
| Anti-HLA I | QVQLKQSGPGLVQPSQSLSLTC | SIVMTQTPKFLLVSAGDRVTITCKA |
| K409R | TVSGFSLTSYGVHWVRQPPGK | SQSVSNDVAWYQQKPGQSPKLLIY |
| LALAPG | GLEWLGVIWSGGSTDYN | YASNRYTGVPD |
| | AAFISRLSIRKDNSKSQVFFKMN | RFTGSGYGTDFTFTISTVQAEDLAV |
| | SLQADDTAIYYCARTFTTSTSAW | YFCQQDYSSPPWTFGGGTKLEIRR |
| | FAYWGQGTLVTVSA | TVAAPSVFIFP |
| | ASTKGPSVFPLAPSSKSTSGGT | PSDEQLKSGTASVVCLLNNFYPRE |
| | AALGCLVKDYFPEPVTVSWNSG | AKVQWKVDNALQSGNSQESVTEQ |
| | ALTSGVHTFPAVLQSS | DSKDSTYSLSSTL |
| | GLYSLSSVVTVPSSSLGTQTYIC | TLSKADYEKHKVYACEVTHQGLSS |
| | NVNHKPSNTKVDKRVEPKSCDK | PVTKSFNRGEC* (SEQ ID NO: 14) |
| | THTCPPCPAPEAAGG | |
| | PSVFLFPPKPKDTLMISRTPEVT | |
| | CVVVDVSHEDPEVKFNWYVDG | |
| | VEVHNAKTKPREEQYN | |
| | STYRVVSVLTVLHQDWLNGKEY | |
| | KCKVSNKALGAPIEKTISKAKGQ | |
| | PREPQVYTLPPSREE | |
| | MTKNQVSLTCLVKGFYPSDIAVE | |
| | WESNGQPENNYKTTPPVLDSD | |
| | GSFFLYS [symbol] LTVDKSRW | |
| | QQGNVFSCSVMHEALHNHYTQ | |
| | KSLSLSPGK* (SEQ ID NO: 13) | |

TABLE 2
Amino acid sequences for bispecific antibodies.

| Bispecific | | | | |
|---|---|---|---|---|
| Antibody | Heavy chain | Light chain | Heavy chain | Light chain |
| Anti-FH F405L | SEQ ID NO: 1 | SEQ ID NO: 2 | SEQ ID NO: 5 | SEQ ID NO: 6 |
| LALAPG, Anti- | | | | |
| DNP K409R | | | | |
| LALAPG | | | | |
| Anti-FH F405L | SEQ ID NO: 1 | SEQ ID NO: 2 | SEQ ID NO: 9 | SEQ ID NO: 10 |
| LALAPG, Anti- | | | | |
| HIV K409R | | | | |
| LALAPG | | | | |
| Anti-FH F405L | SEQ ID NO: 1 | SEQ ID NO: 2 | SEQ ID NO: 11 | SEQ ID NO: 12 |
| LALAPG, Anti- | | | | |
| biotin K409R | | | | |
| LALAPG | | | | |
| Anti-FH F405L | SEQ ID NO: 1 | SEQ ID NO: 2 | SEQ ID NO: 13 | SEQ ID NO: 14 |
| LALAPG, Anti- | | | | |
| HLA I K409R | | | | |
| LALAPG | | | | |
| Anti-C4BP | SEQ ID NO: 3 | SEQ ID NO: 4 | SEQ ID NO: 5 | SEQ ID NO: 6 |
| F405L LALAPG, | | | | |
| Anti-DNP | | | | |
| K409R LALAPG | | | | |
| Anti-C4BP | SEQ ID NO: 3 | SEQ ID NO: 4 | SEQ ID NO: 9 | SEQ ID NO: 10 |
| F405L LALAPG, | | | | |
| Anti-HIV K409R | | | | |
| LALAPG | | | | |
| Anti-C4BP | SEQ ID NO: 3 | SEQ ID NO: 4 | SEQ ID NO: 11 | SEQ ID NO: 12 |
| F405L LALAPG, | | | | |
| Anti-biotin | | | | |
| K409R LALAPG | | | | |
| Anti-C4BP | SEQ ID NO: 3 | SEQ ID NO: 4 | SEQ ID NO: 13 | SEQ ID NO: 14 |
| F405L LALAPG, | | | | |
| Anti-HLA I | | | | |
| K409R LALAPG | | | | |
| Anti-HIV F405L | SEQ ID NO: 7 | SEQ ID NO: 8 | SEQ ID NO: 5 | SEQ ID NO: 6 |
| LALAPG, Anti- | | | | |
| DNP K409R | | | | |
| LALAPG | | | | |
| Anti-HIV F405L | SEQ ID NO: 7 | SEQ ID NO: 8 | SEQ ID NO: 9 | SEQ ID NO: 10 |
| LALAPG, Anti- | | | | |
| HIV K409R | | | | |
| LALAPG | | | | |
| Anti-HIV F405L | SEQ ID NO: 7 | SEQ ID NO: 8 | SEQ ID NO: 11 | SEQ ID NO: 12 |
| LALAPG, Anti- | | | | |
| biotin K409R | | | | |
| LALAPG | | | | |
| Anti-HIV F405L | SEQ ID NO: 7 | SEQ ID NO: 8 | SEQ ID NO: 13 | SEQ ID NO: 14 |
| LALAPG, Anti- | | | | |
| HLA I K409R | | | | |
| LALAPG | | | | |

TABLE 3
Amino acid sequence identifiers of the heavy chain complementarity-determining
regions (CDRs) and light chain complementarity-determining regions (CDRs).

| | HC CDR1 | HC CDR2 | HC CDR3 | LC CDR1 | LC CDR2 | LC CDR3 |
|---|---|---|---|---|---|---|
| Anti-FH | GYSFTGY | INPYNGA | ARGLFLD | QGITSN | HG | VQYAQFPWT |
| F405L | F | T | Y | (SEQ ID | (SEQ ID | (SEQ ID NO: |
| LALAP | (SEQ ID | (SEQ ID | (SEQ ID | NO: 18) | NO: 19) | 20) |
| G | NO: 15) | NO: 16) | NO: 17) | | | |
| Anti- | GFIFSDY | ISDGGVY | VRGDYY | QSLLDGD | LV | CQGTHFPWT |
| C4BP | Y | T | GNSLDY | GKTY | (SEQ ID | (SEQ ID NO: |
| F405L | (SEQ ID | (SEQ ID | (SEQ ID | (SEQ ID | NO: 25) | 26) |
| LALAP | NO: 21) | NO: 22) | NO: 23) | NO: 24) | | |
| G | | | | | | |
| Anti- | GYSITNS | IGYDGSN | ARATYYG | QDISNY | YT | QQGNTLPWT |
| DNP | YY | (SEQ ID | NYRGFA | (SEQ ID | (SEQ ID | (SEQ ID NO: |
| K409R | (SEQ ID | NO: 28) | Y | NO: 30) | NO: 31) | 32) |
| LALAP | NO: 27) | | (SEQ ID | | | |
| G | | | NO: 29) | | | |
| Anti- | GYRFSNF | INPYNGN | ARVGPY | HSIRSRR | GV | QVYGASSYT |
| HIV | V | K | SWDDSP | (SEQ ID | (SEQ ID | (SEQ ID NO: |
| F405L | (SEQ ID | (SEQ ID | QDNYYM | NO: 36) | NO: 37) | 38) |
| LALAP | NO: 33) | NO: 34) | DV | | | |
| G | | | (SEQ ID | | | |
| | | | NO: 35) | | | |
| Anti- | GYRFSNF | INPYNGN | ARVGPY | HSIRSRR | GVS | QVYGASSYT |
| HIV | V | K | SWDDSP | (SEQ ID | (SEQ ID | FGQGKLERK |
| K409R | (SEQ ID | (SEQ ID | QDNYYM | NO: 42) | NO: 43) | (SEQ ID NO: |
| LALAP | NO: 39) | NO: 40) | DVWGKG | | | 44) |
| G | | | TTVIVSS | | | |
| | | | (SEQ ID | | | |
| | | | NO: 41) | | | |
| Anti- | GFSLTAY | IWGGGR | VKHTNW | QGLVYS | KV | SQSTHFPFT |
| biotin | G | T | DGGFAY | NGDTY | (SEQ ID | (SEQ ID NO: |
| K409R | (SEQ ID | (SEQ ID | (SEQ ID | (SEQ ID | NO: 49) | 50) |
| LALAP | NO: 45) | NO: 46) | NO: 47) | NO: 48) | | |
| G | | | | | | |
| Anti- | GFSLTSY | IWSGGST | ARTFTTS | QSVSND | YA | QQDYSSPP |
| HLA I | G | (SEQ ID | TSAWFA | (SEQ ID | (SEQ ID | WT |
| K409R | (SEQ ID | NO: 52) | Y | NO: 54) | NO: 55) | (SEQ ID NO: |
| LALAP | NO: 51) | | (SEQ ID | | | 56) |
| G | | | NO: 53) | | | |

### 3B9D6 and OX-24 Hybridomas

Hybridomas producing antibodies directed against human FH were acquired from ECACC (OX-24). Hybridomas producing antibodies directed against human C4BP were acquired from Podiceps BV, Utrecht, the Netherlands (3B9D6). The hybridomas were cultured and used to generate recombinant anti-FH and anti-C4BP antibodies, respectively. Hybridoma cells were cultured in Roswell Park Memorial Institute (RPMI) 1640 medium (Gibco) supplemented with penicillin, streptomycin, 10% fetal calf serum (FCS), 2 mM L-glutamine (all Gibco), and 50 μM β-mercaptoethanol (Sigma-Aldrich) at 37° C. in 5% CO₂.

### Sequencing

RNA was isolated from hybridoma cells using TRIzol reagent (Thermo Fisher Scientific), chloroform, isopropyl alcohol, and ethanol (all Merck). Next, SMART cDNA synthesis was performed using PrimeScript Reverse Transcriptase (Takara), and VH and VL gene products were amplified by 5′-RACE polymerase chain reaction (PCR). The VH and VL PCR products were treated with Taq polymerase (Promega) and afterwards purified from excised bands using the Zymoclean gel DNA recovery kit (Zymo Research). Subsequently, the VH and VL products were cloned into pcDNA3.3 expression vectors using the pcDNA3.3-TOPO TA cloning kit (Thermo Fisher Scientific). The vectors were used for transformation of One Shot TOP10 E. coli competent cells (Thermo Fisher Scientific) by heat shock. The transformed cells were cultured on LB agar plates supplemented with 100 μg/mL ampicillin (Sigma-Aldrich) and after overnight incubation at 37° C., multiple single colonies were picked and grown overnight in LB medium containing ampicillin. From the cultures, plasmids were isolated using the QIAprep spin miniprep kit (Qiagen). The plasmids were sequenced by the Leiden Genome Technology Center (LGTC) to obtain the VH and VL sequences.

### DNA Constructs

DNA constructs consisting of the Hindlll restriction site (AAGCTT), a Kozak sequence (GCCGCCACC), a signal peptide [MEFGLSWVFLVALLRGVQC (SEQ ID NO: 57)], the VH/VL sequence, and the Apal restriction site (GGGCCC, in case of VH) or the BsiWI restriction site (CGTACG, in case of VL) were designed. DNA constructs were ordered as a DNA fragment or in a DNA vector (GeneArt—Thermo Fisher Scientific). DNA constructs were digested with Hindlll and Apal or BsiWI and ligated into a pcDNA3.3 expression vector containing the constant domain of human IgG1 (IGHG1*03) or K (IGKC). Depending on the specificity, different mutations were introduced. Anti-CD20 and anti-CD52 were kept wild-type (IgG1K). Anti-FH and anti-C4BP were designed with the F405L mutation, whereas anti-DNP, anti-biotin, and anti-HLA I were designed with the K409R mutation (mutations necessary for Fab-arm exchange, see below). Anti-HIV was designed with either the F405L mutation or the K409R mutation. All antibodies except anti-CD20 and anti-CD52 contained LALAPG mutations to make their Fc functionally inactive (7).

### Transfection

For antibody production, heavy and light chain containing vectors were used for transient co-transfection of Expi293F cells with ExpiFectamine, Opti-MEM, and Expi293 expression medium (Thermo Fisher Scientific) according to the instructions provided by the manufacturer. After 5 days of culture, supernatants containing the antibodies were harvested and filtered.

### Antibody Purification (Protein A)

For antibody purification, filtered supernatants were loaded onto a column packed with protein A resin (GenScript). After washing the column, antibodies were eluted with 20 mM glycine pH 2.8 and neutralized with 1 M Tris HCl pH 9.5. Antibodies were concentrated and buffer exchanged to PBS using Ultra Centrifugal filter units. Concentrations of purified antibodies were determined by NanoDrop.

### Fab-Arm Exchange

The generation of bsAbs was done by Fab-arm exchange as described by Labrijn and colleagues (6). The complement regulator (FH/C4BP)-binding parental antibodies had the F405L-LALAPG mutations and the target (DNP/biotin/HLA I)-binding parental antibodies had the K409R-LALAPG mutations. The anti-HIV arm served as a non-binding arm (isotype control) to preserve the bsAb architecture. The quality of the bsAbs was confirmed via bispecificity ELISA and mass spectrometry (see below).

### Example 2. Quality of Bispecific Antibodies

It was confirmed that the bsAbs can bind to a site-specific target with one arm and to the endogenous complement regulators FH or C4BP with the other arm. Monospecific antibodies were generated bearing LALAPG (SEQ ID NO: 58) mutations in the Fc region to block C1q and FcγR binding (7), making them unable to recruit additional effector functions. In addition, the Fc region contained additional complementary mutations that allow the generation of bsAbs using the procedure of controlled Fab-arm exchange (6).

### Bispecific ELISA

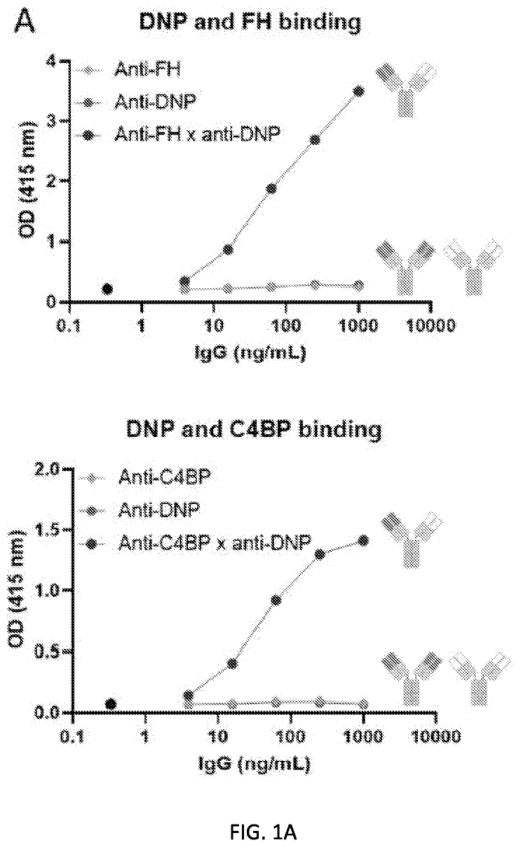

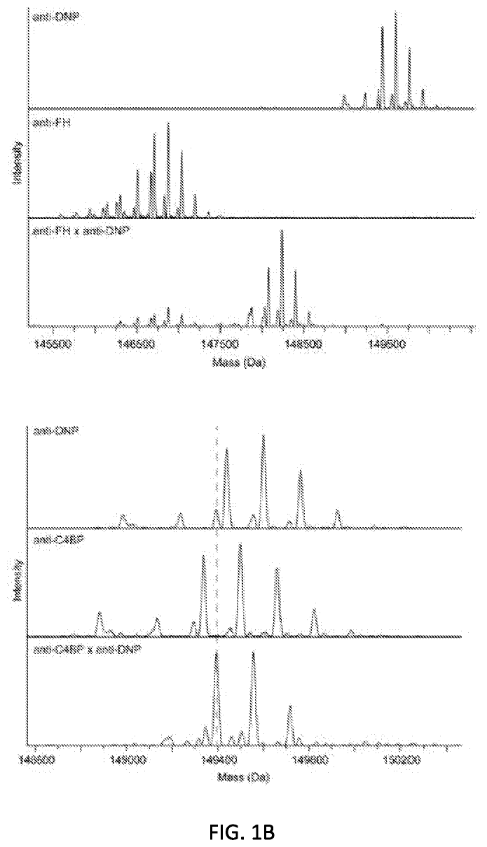

BsAbs were tested for binding to both antigens in a bispecific ELISA. MaxiSorp plates (Thermo Scientific, Nunc, 430341) were coated with 50 μL/well BSA-DNP (10 μg/mL, Biosearch Technologies, D-5050-10, conjugation ration 13) in coating buffer (0.1 M Na₂CO₃/NaHCO₃PH 9.6) for 1 hour at 37° C. After every incubation, plates were washed three times with PBS/0.05% Tween 20. Plates were blocked with 100 μL/well PBS/1% BSA for 1 hour at 37° C. After washing, plates were incubated with 50 μL/well (bs) Abs diluted in PBS/0.05% Tween 20/1% BSA (PTB buffer) for 1 hour at 37° C. Next, 1 μg/mL FH (CompTech, A137) or C4BP (CompTech, A109) diluted in PTB buffer was added. FH was detected using goat anti-human FH (Quidel, A312) and HRP-labeled rabbit anti-goat Ig (Dako, P0449), and C4BP was detected using rabbit anti-human C4BP (kind gift from Anna M. Blom, Malmö, Sweden) and HRP-labeled goat anti-rabbit Ig (Dako, P0448), all diluted in PTB buffer. Plates were developed by incubating with 50 μL/well ABTS (Merck, A1888-5G) containing 1:2000 diluted H₂O₂ (Merck, 1072090250), and read at 415 nm using a microplate reader (BIO-RAD iMark). It was confirmed by ELISA that the bsAbs can bind to a specific target (e.g., DNP as a model antigen) as well as FH or C4BP, whereas the parental monospecific antibodies cannot (FIG. 1A).

### Mass Spectrometry

Intact mass analysis of parental and bsAbs was performed using sheathless capillary electrophoresis (CESI 8000 instrument, Sciex) hyphenated to an Impact QTOF mass spectrometer (Bruker Daltonics). A bare-fused silica capillary containing a porous tip (91 cm×30 μm ID, Sciex) was used for the analyses. Prior to the experiments, the capillary was coated with polyethylenimine (Gelest) following the protocol described by Sciex (13). As background electrolyte (BGE) 10% acetic acid was employed. Prior to each run, the capillary was flushed for 3 minutes at 100 psi (forward and reverse pressure) with the BGE. Before analysis, samples were buffer exchanged to 10 mM acetate adjusted with ammonium acetate to pH 3.1, using 30 kDa MWCO filters (Vivaspin, 3 cycles of 10000×g at 4° C.). Samples were injected hydrodynamically by applying 2.5 psi for 15 seconds. Separation was carried out applying a voltage of −20 KV at 20° C. The mass spectrometer was operated in positive ionization mode using a capillary voltage of 1100 V, a drying gas temperature of 120° C., and a flow of 1.2 L/minute. An in-source CID energy of 100 eV was used for declustering. To further enhance declustering, the quadrupole and collision cell energy were set at 5.0 and 20.0 eV, respectively. For data analysis, the Data Analysis software (Bruker) was used. To determine the intact mass of the bs(Abs), the mass spectra were deconvoluted using the Maximum Entropy deconvolution algorithm. A baseline subtraction of 0.8 points was applied on the obtained zero deconvoluted spectra.

Using native mass spectrometry it was confirmed that the masses of the bsAbs are in between the masses of the parental monospecific antibodies (FIG. 1B), showing that pure fractions of bsAbs were successfully generated. Although for some of the parental monospecific antibodies the masses were rather similar, it was still possible to confirm that the fractions of bsAbs were pure (FIG. 1B, bottom). For the subsequent functional analyses, all bsAbs were compared with control bsAbs in which either the target antigen-binding arm or the complement regulator-binding arm was replaced by an antibody arm binding HIV gp120 (b12).

### Example 3. Targeted Bispecific Antibodies that Bind Endogenous Complement Inhibitors Decrease Complement Activation

### Complement Activation Assay

BsAbs were functionally tested in plate-bound complement activation assays. To focus on the classical pathway, MaxiSorp plates were coated with 50 μL/well of a mixture of IgG (10 μg/mL, IVIg, Sanquin) or IgM (1 μg/mL, Merck, 401799) and BSA-DNP (10 μg/mL) or biotinylated BSA-DNP (10 μg/mL, biotinylated in-house) in coating buffer for 1 hour at 37° C. After every incubation, plates were washed three times with PBS/0.05% Tween 20. Plates were blocked with 100 μL/well PBS/1% BSA for 1 hour at 37° C. Samples containing 5 μg/mL (or a titration from 0.1 to 10 μg/mL) bsAbs, 10 μg/mL FH or C4BP, and 1% normal human serum (NHS) in RPMI were preincubated for 30 minutes at 4° C. After washing, plates were incubated with 50 μL/well samples for 1 hour at 37° C. Next, FH and C4BP were detected as described above for the bispecificity ELISA (using goat anti-human FH and HRP-labeled rabbit anti-goat Ig, or using rabbit anti-human C4BP and HRP-labeled goat anti-rabbit Ig), and C5b-9 was detected using mouse anti-human C5b-9 (Dako, M0777) and HRP-labeled goat anti-mouse Ig (Dako, P0447), all diluted in PTB buffer. Plates were developed by incubating with 50 μL/well ABTS containing 1:2000 diluted H₂O₂, and read at 415 nm using a microplate reader.

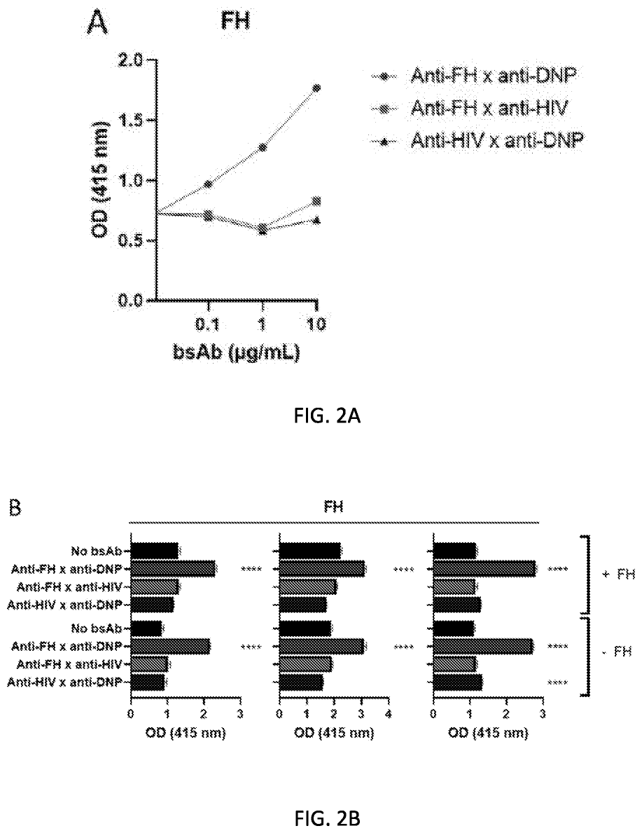

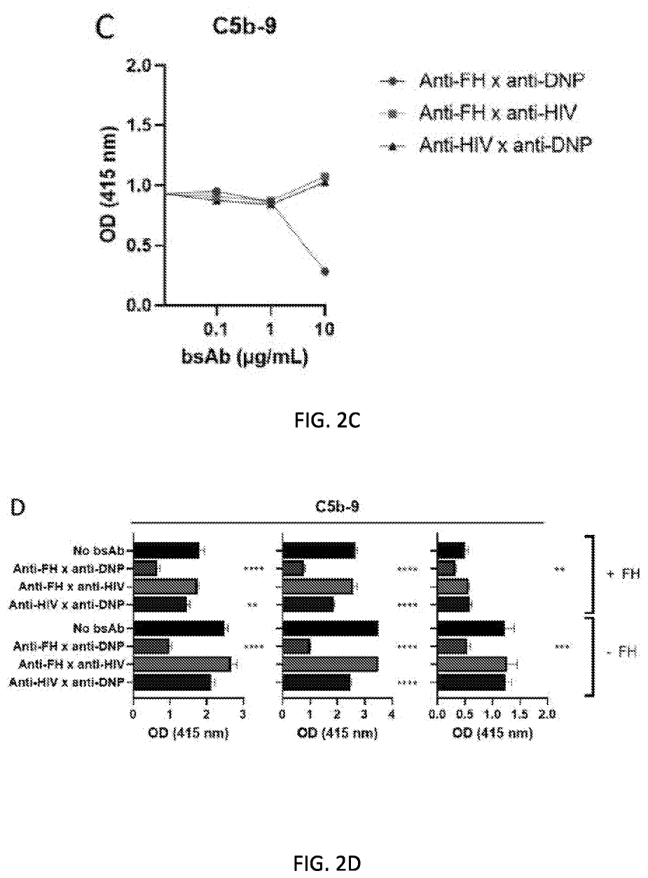

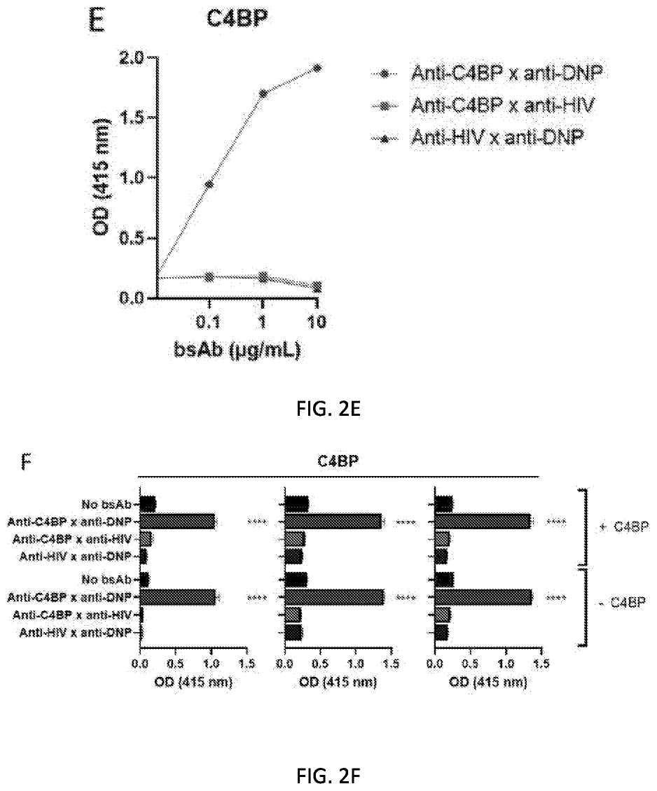

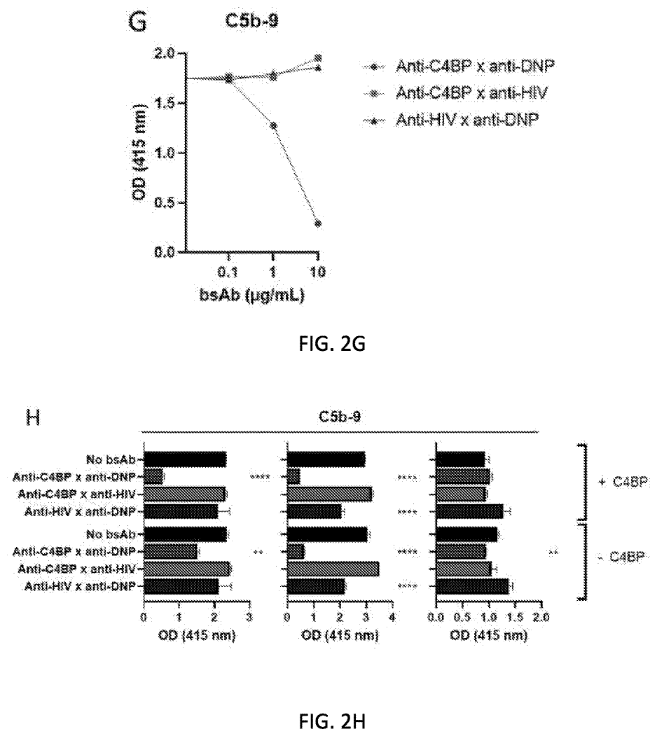

To focus on the lectin pathway, a similar assay was performed, but plates were coated with a mixture of acetylated HSA (10 μg/mL, acetylated in-house) and BSA-DNP. In addition to NHS, C1q- and factor B-depleted serum were used, all at 3%. To focus on the alternative pathway, plates were coated with a mixture of LPS (10 μg/mL, Sigma-Aldrich, L2012-10 MG) and BSA-DNP. Besides, samples contained more NHS (10%) and were prepared in RPMI-MgEGTA. The functional effects of the bsAbs were tested in plate-bound complement activation assays. A specific target (DNP) was coated to allow binding of the bsAbs, together with IgG to activate the classical pathway, acetylated HSA to activate the lectin pathway, or LPS to activate the alternative pathway of the complement system. Upon incubation with normal human serum (NHS), an increase in FH presence was detected for the bsAb binding both FH and DNP, whereas for the control bsAbs (binding either FH or DNP) FH did not increase (FIG. 2A,B). This increase in FH was associated with a decrease in C5b-9 (FIG. 2C,D), showing that the bsAb binding both FH and DNP inhibits complement activation as it decreases membrane attack complex formation (C5b-9). Adding exogenous FH to NHS (which at 1% contains ~3-5 μg/mL FH) was not necessary and did not improve the complement-inhibiting effect, indicating that FH endogenously present in human serum is sufficient to mediate the desired effects (FIG. 2D).

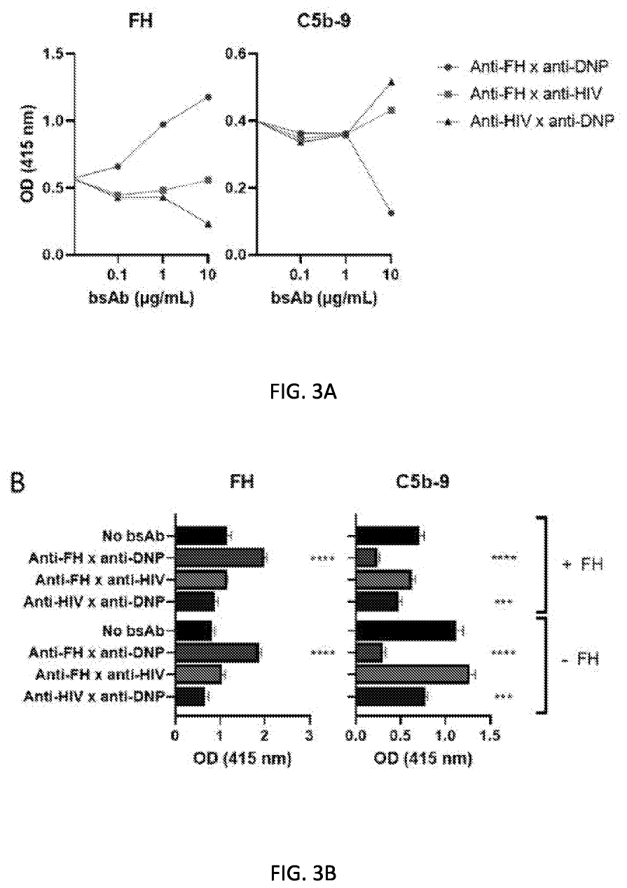

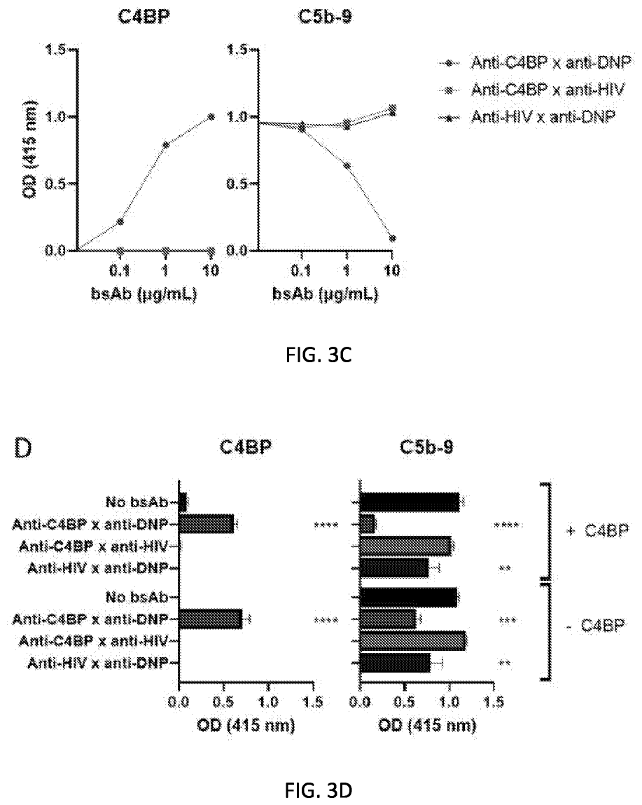

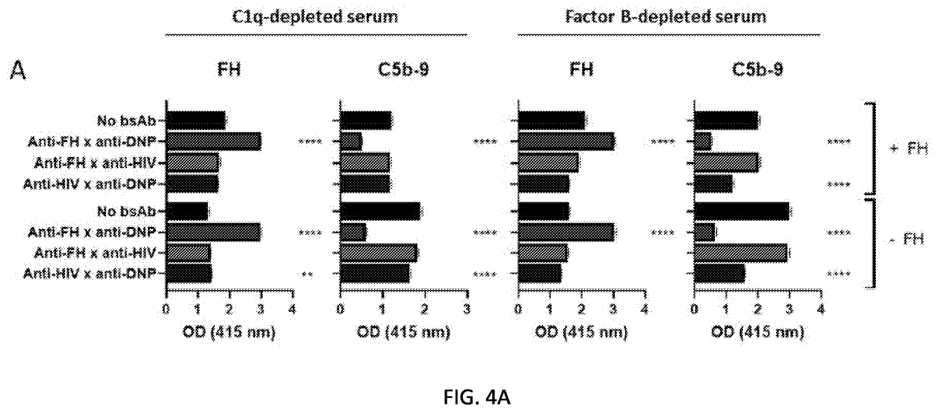

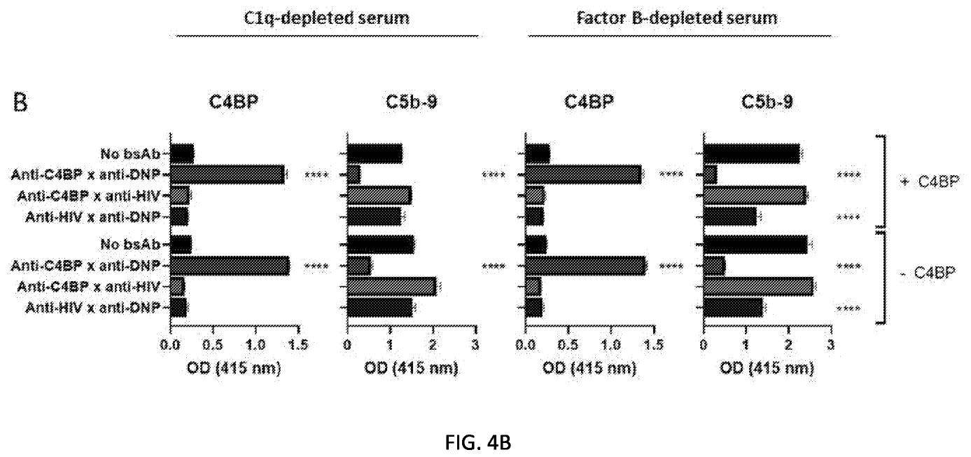

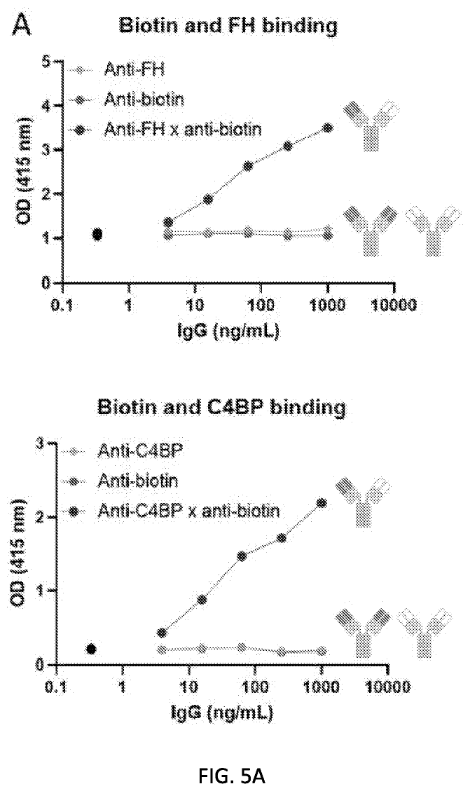

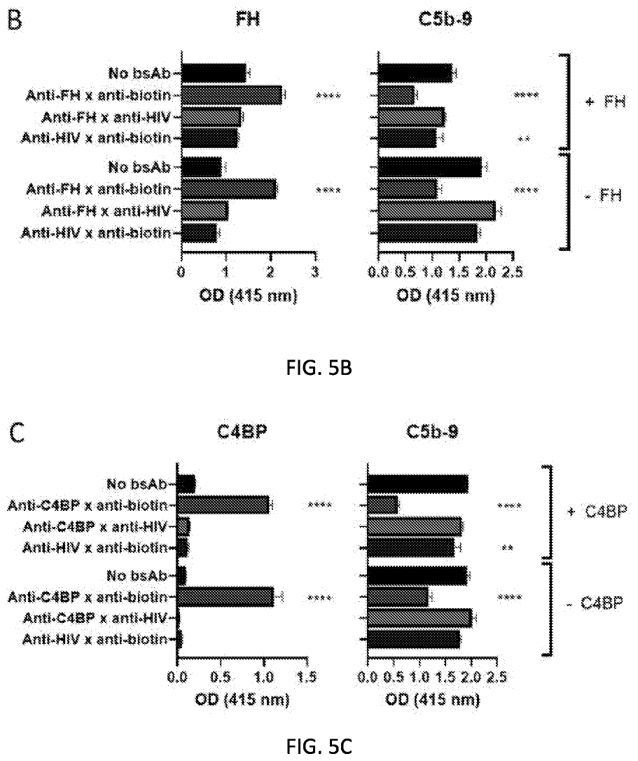

For the bsAb binding both C4BP and DNP, an increase in C4BP presence was detected upon incubation with NHS, whereas for the control bsAbs (binding either C4BP or DNP) C4BP did not increase (FIG. 2E,F). This increase in C4BP was associated with a decrease in C5b-9 (FIG. 2G,H), showing that similarly to the results above, the bsAb binding both C4BP and DNP also decreases C5b-9 formation and thus inhibits complement activation. Adding exogenous C4BP to NHS (which at 1% contains ~2 μg/mL C4BP) was not necessary, but did improve inhibition of the classical but not the alternative or lectin pathway (FIG. 2H). For the classical pathway, experiments were repeated using another initiating molecule, namely IgM. Very similar results were obtained, confirming that the bsAbs binding both FH and DNP (FIG. 3A,B) or C4BP and DNP (FIG. 3C,D) capture FH or C4BP thereby increasing their local concentration, and decrease C5b-9 formation and thus inhibit complement activation. For the lectin pathway, we repeated our experiments using C1q- or factor B-depleted serum and observed clear inhibition (FIG. 4), indicating that the observed inhibition is not (only) classical or alternative pathway dependent but driven by lectin pathway activity. When using another target instead of DNP, namely biotin, and another bsAb, binding both FH/C4BP and biotin (FIG. 5A), the results were very similar (FIG. 5B,C), showing that the complement-inhibiting effect observed for the set of bsAbs is a general phenomenon and is not dependent on the specific target.

In summary, it was observed that in plate-bound assays the bsAbs were able to inhibit complement activation initiated by the classical, lectin, and alternative pathway. The concentrations of endogenous FH and C4BP were sufficient to mediate local inhibition.

### Example 4. Targeted Bispecific Antibodies Decrease Lysis of Liposomes

Next it was tested whether the bsAbs were also able to protect model-liposomes from complement-mediated lysis.

### Liposome Preparation

Liposomes containing two antigens, mCD52 (peptide with the amino acid sequence TSSPSAD, which is a CD52 mimotope; synthesized by Aimee Boyle, Leiden Institute of Chemistry, Leiden University, Leiden, the Netherlands) and DNP, filled with sulforhodamine B (20 mM; S1402 from Sigma-Aldrich, Missouri, USA) in PBS were produced. Lipid films were composed of dimyristoylphosphatidylcholine (DMPC), dimyristoylphosphatidylglycerol (DMPG), cholesterol, DNP-cap-PE and mCD52-cholesterol (44:5:49:1:1 mol %). Liposomes were prepared as described previously (14-16). All lipids, except mCD52-cholestrol, were purchased from Avanti Polar Lipids (Alabama, USA).

### Liposome Assay

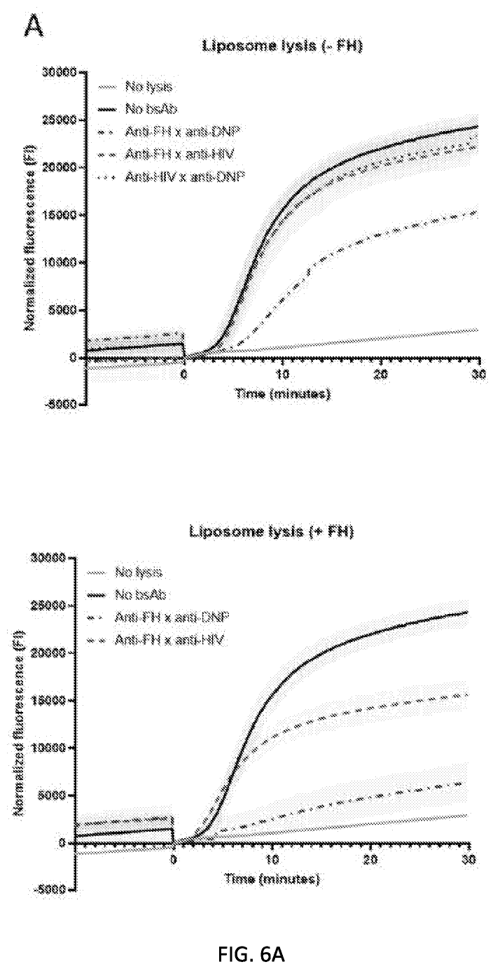

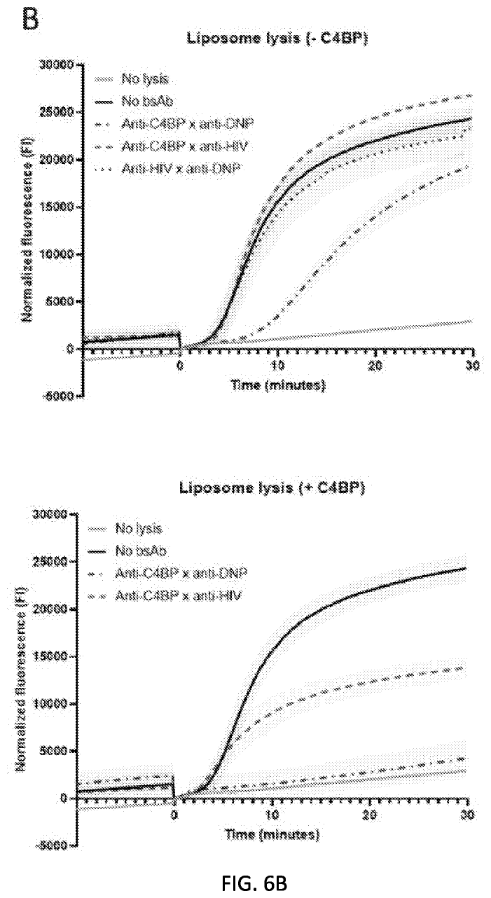

The effect of the bsAbs (anti-FH/C4BP x anti-DNP and controls) on the classical pathway was analyzed in a fluorescence-based complement activation assay using liposomes. Therefore, purified liposomes were incubated with bsAbs (50 μg/mL; 333.3 nM final concentration), NHS (1% v/v final concentration), and with or without either FH (100 μg/mL; 645.2 nM final concentration) or C4BP (100 μg/mL; 200 nM final concentration) for 1 hour at 4° C., before sulforhodamine B fluorescence was measured at a CLARIOstar microplate reader (BMG LABTECH, Offenburg, Germany) with an excitation wavelength of 565 nm and emission wavelength of 585 nm at 21° C. After measuring the fluorescence intensity for 15 minutes, anti-CD52 wild-type (50 μg/mL; 333.3 nM final concentration) was added and fluorescence was measured for another 30 minutes. For the positive control, liposomes were incubated with NHS (without bsAbs) before anti-CD52 wild-type was added. For the negative control, liposomes were incubated with NHS (without bsAbs) and no anti-CD52 wild-type was added. Liposomes containing CD52 and DNP were incubated with the bsAbs (binding both FH/C4BP and DNP) and NHS with or without exogenous FH or C4BP. Upon adding anti-CD52 antibodies to activate the classical pathway of the complement system, lysis of liposomes was clearly decreased by the bsAb binding both FH and DNP and not by the control bsAbs (binding either FH or DNP) (FIG. 6A). Adding exogenous FH to NHS already decreased liposome lysis in the presence of the control bsAb binding only FH, but importantly a nearly complete block in liposome lysis was obtained in the presence of the bsAb binding both FH and DNP (FIG. 6A). This shows that fluid-phase FH can decrease liposome lysis, but FH targeted to the liposomes by our bsAb is much more effective in protecting liposomes from complement-mediated lysis, thereby providing important proof for our concept. Very similar results were obtained with the bsAb binding both C4BP and DNP, which with adding exogenous C4BP to NHS almost completely prevented liposome lysis (FIG. 6B). Overall, the data demonstrate that the bsAbs protect model-liposomes from complement-mediated lysis.

### Example 5. Targeted Bispecific Antibodies Protect Erythrocytes from Complement-Mediated Lysis

It was investigated whether the bsAbs could protect erythrocytes from lysis in classical and alternative pathway assays. To analyze the impact on classical pathway-mediated lysis, sheep red blood cells were sensitized with rabbit anti-sheep wild-type antibodies. These cells were biotinylated and treated with bsAbs (anti-FH/C4BP x anti-biotin). To analyze the impact on the alternative pathway, rabbit red blood cells (which lack endogenous FH binding) were biotinylated and treated with bsAbs (anti-FH/C4BP x anti-biotin).

### Hemolytic Assay

To study the effect of the targeted bsAbs on activity of the classical pathway, (rabbit anti-sheep wild-type) antibody-sensitized sheep red blood cells (Sanquin) were washed three times with PBS and biotinylated (Thermo Scientific, 21331) for 30 minutes at room temperature. After washing two times with PBS, cells were incubated with 10 μg/mL (or a titration from 0.1 to 10 μg/mL) bsAbs (anti-FH/C4BP x anti-biotin and controls) with or without 10 μg/mL FH or C4BP diluted in PBS for 30 minutes at room temperature. After washing, cells were incubated with 0.5% NHS in RPMI for 1 hour at 37° C. For the positive control, cells were incubated with water to get 100% lysis. Cells were centrifuged and supernatants were transferred to MaxiSorp plates, which were read at 405 nm using a microplate reader.

To study the effect of the targeted bsAbs on activity of the alternative pathway, a similar assay was performed, but rabbit red blood cells (which lack endogenous FH binding, Sanquin) were used and incubated with 5% NHS in RPMI-MgEGTA.

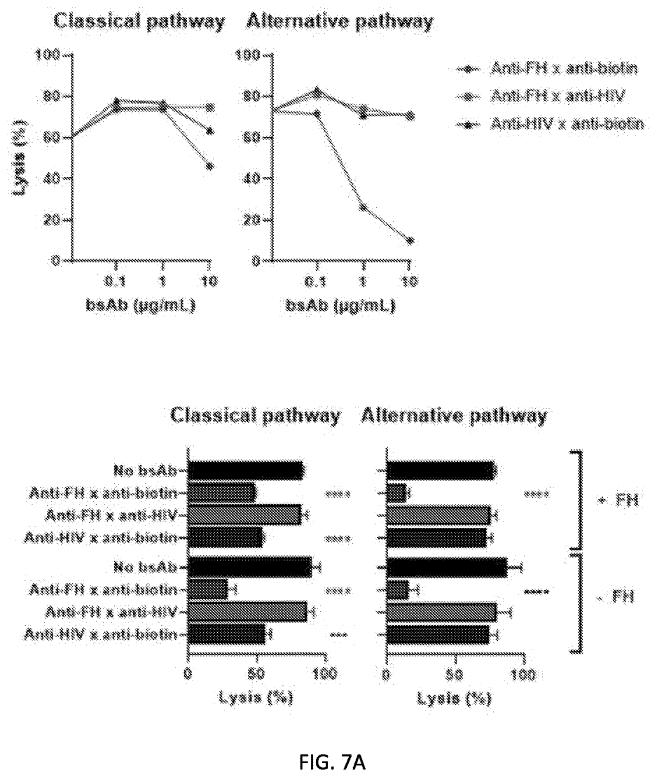

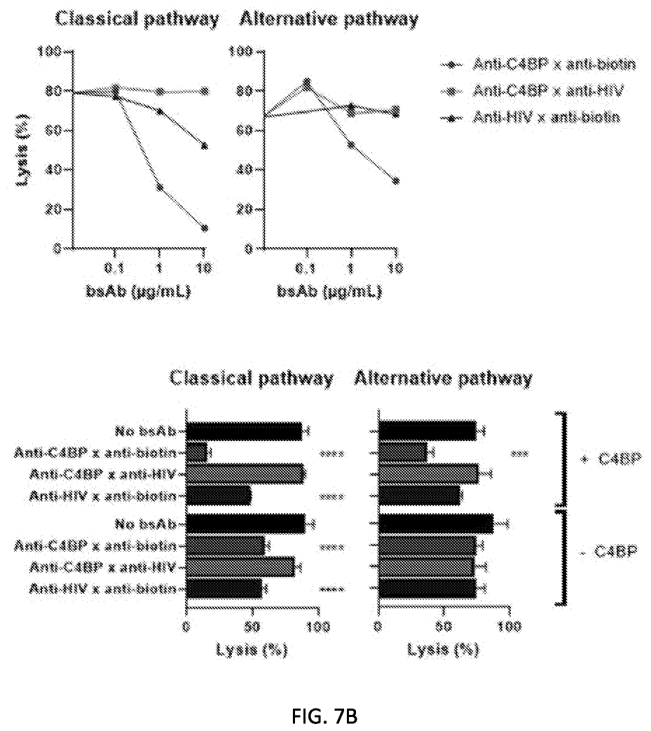

Upon incubation with NHS, the bsAb binding both FH and biotin clearly inhibited lysis of red blood cells, whereas the control bsAbs did not (FIG. 7A). Since FH is the main fluid-phase regulator of the alternative pathway, it was noted that the effect on alternative pathway-mediated lysis is bigger than the effect on classical pathway-mediated lysis. Adding exogenous FH to NHS was not necessary and did not improve the complement-inhibiting effect.

The bsAb binding both C4BP and biotin (and not the control bsAbs) also clearly decreased lysis of red blood cells (FIG. 7B). Since C4BP is the main fluid-phase regulator of the classical pathway, it was to be expected that the effect on classical pathway-mediated lysis is larger than the effect on alternative pathway-mediated lysis. For the classical pathway, adding exogenous C4BP to NHS was not necessary, but did improve the complement-inhibiting effect. However, for the alternative pathway, adding exogenous C4BP to NHS was necessary to observe a complement-inhibiting effect.

In summary, the bsAbs were also able to protect erythrocytes from lysis in classical and alternative pathway assays, where the FH-binding bsAb was most effective in alternative pathway-mediated lysis and the C4BP-binding bsAb was most effective in classical pathway-mediated lysis.

### Example 6. Targeted Bispecific Antibodies Protect White Blood Cells from Complement-Mediated Cytotoxicity

It was investigated whether the bsAbs were able to protect human leukocytes from complement-mediated lysis.

### CDC Assay

Ramos cells (originally from ATCC) were plated at 100,000 cells/well (V-bottom microplate, Greiner Bio-One, 651101). Cells were incubated with 100 μL/well samples containing 0.3 μg/mL anti-CD20 wild-type with or without 20 μg/mL bsAbs (anti-FH x anti-HLA I and controls) with or without 10 μg/mL FH for 30 minutes. After every incubation, cells were washed with 150 μL/well PBS/1% FCS. Cells were incubated with 100 μL/well 10% NHS in RPMI (for FH detection in the presence of 50 μg/mL eculizumab) for 45 minutes at 37° C. Cell viability was measured by adding 100 μL/well 7-AAD (BD Pharmingen, 559925) 1:200 diluted in PBS/1% FCS. FH was detected using mouse anti-human FH (1:100, OX-23, generated in-house) and goat anti-mouse IgG AF488 (1:50, Life Technologies, A11001). Cells were measured using a FACSCanto flow cytometer (BD Biosciences).

A similar assay was performed with thawed peripheral blood mononuclear cells (PBMCs, isolated from healthy donors using Ficoll) instead of cultured Ramos cells. For PBMCs, 1 μg/mL anti-CD52 wild-type instead of anti-CD20 wild-type was used to kill cells, and a higher concentration of bsAbs (30 μg/mL) was used.

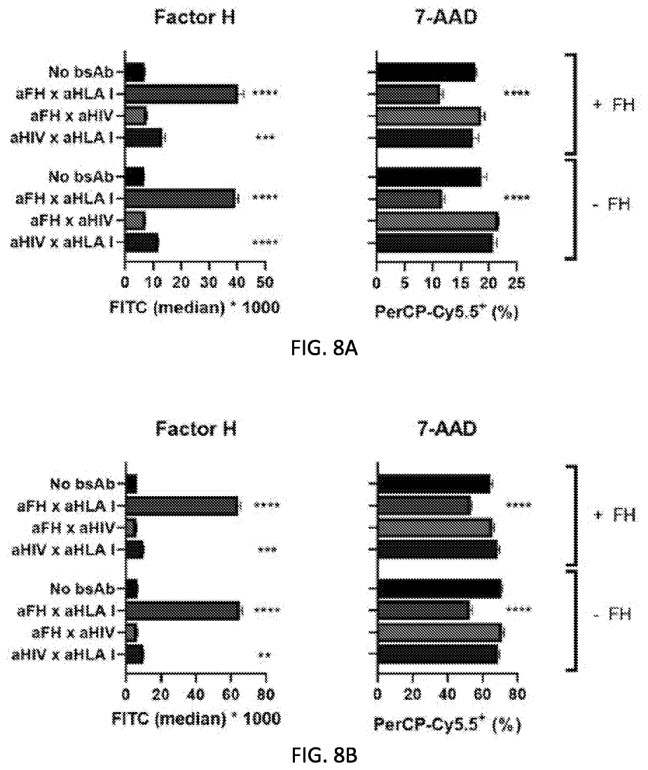

First, Ramos cells, a B-cell line expressing CD20, and complement-mediated lysis of these cells was triggered by incubating these cells with anti-CD20 antibodies and NHS. It was studied if targeted inhibition of complement activation could be achieved by using a bsAb that binds human leukocyte antigen class I (HLA I), a target antigen expressed on the surface of all nucleated cells. The bsAb binding both FH and HLA I was able to target FH to the cells, which resulted in protection from lysis (FIG. 8A). Adding exogenous FH to NHS was not necessary and did not further improve the complement-inhibiting effect.

To confirm this complement-inhibiting effect, a cell line was switched to peripheral blood mononuclear cells (PBMCs). Since CD52 is expressed on the surface of most PBMCs, cells were sensitized with anti-CD52 antibodies to activate the classical pathway of the complement system upon adding NHS. The bsAb binding both FH and HLA I was able to target FH to the cells, which resulted in protection from lysis (FIG. 8B). Adding exogenous FH to NHS was not necessary and did not clearly improve the complement-inhibiting effect.

In summary, the bsAbs were able to locally inhibit complement activation initiated by the classical, lectin, and alternative pathway in plate-bound assays. The bsAbs were also able to protect model-liposomes from complement-mediated lysis as well as erythrocytes in classical and alternative pathway assays. Finally, the bsAbs were able to protect human leukocytes from complement-mediated lysis.

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Sequences
(SEQ ID NO: 57)
MEFGLSWVFLVALLRGVQC
(SEQ ID NO: 58)
LALAPG

### REFERENCES

1. M. Mullazehi, L. Mathsson, J. Lampa, J. Ronnelid, High anti-collagen type-II antibody levels and induction of proinflammatory cytokines by anti-collagen antibody-containing immune complexes in vitro characterise a distinct rheumatoid arthritis phenotype associated with acute inflammation at the time of disease onset. Ann Rheum Dis 66, 537-541 (2007).
2. S. P. McAdoo, C. D. Pusey, Anti-Glomerular Basement Membrane Disease. Clin J Am Soc Nephrol 12, 1162-1172 (2017).
3. J. J. Verschuuren et al., Pathophysiology of myasthenia gravis with antibodies to the acetylcholine receptor, muscle-specific kinase and low-density lipoprotein receptor-related protein 4. Autoimmun Rev 12, 918-923 (2013).
4. J. M. Thurman, S. E. Panzer, M. Le Quintrec, The role of complement in antibody mediated transplant rejection. Mol Immunol 112, 240-246 (2019).
5. J. Dobo, A. Kocsis, P. Gal, Be on Target: Strategies of Targeting Alternative and Lectin Pathway Components in Complement-Mediated Diseases. Front Immunol 9, 1851 (2018).
6. A. F. Labrijn et al., Controlled Fab-arm exchange for the generation of stable bispecific IgG1. Nat Protoc 9, 2450-2463 (2014).
7. M. Lo et al., Effector-attenuating Substitutions That Maintain Antibody Stability and Reduce Toxicity in Mice. J Biol Chem 292, 3900-3908 (2017).
8. F. Kohen et al., Preparation and properties of anti-biotin antibodies. Methods Enzymol 279, 451-463 (1997).
9. J. S. Crowe, V. S. Hall, M. A. Smith, H. J. Cooper, J. P. Tite, Humanized monoclonal antibody CAMPATH-1H: myeloma cell expression of genomic constructs, nucleotide sequence of cDNA constructs and comparison of effector mechanisms of myeloma and Chinese hamster ovary cell-derived material. Clin Exp Immunol 87, 105-110 (1992).
10. M. L. Gonzalez, M. B. Frank, P. A. Ramsland, J. S. Hanas, F. J. Waxman, Structural analysis of IgG2A monoclonal antibodies in relation to complement deposition and renal immune complex deposition. Mol Immunol 40, 307-317 (2003).
11. C. F. Barbas, 3rd et al., Molecular profile of an antibody response to HIV-1 as probed by combinatorial libraries. J Mol Biol 230, 812-823 (1993).
12. N. Congy-Jolivet, D. Drocourt, S. Portet, G. Tiraby, A. Blancher, Production and characterization of chimeric anti-HLA monoclonal antibodies targeting public epitopes as tools for standardizations of the anti-HLA antibody detection. J Immunol Methods 390, 41-51 (2013).
13. M. R. R. Santos, C. K.; Fonslow, B.; Guttman, A., A covalent, cationic polymer coating method for the CESI-MS analysis of intact proteins and polypeptides. RUO-MKT-18-2325-A.
14. T. H. Sharp et al., Insights into IgM-mediated complement activation based on in situ structures of IgM-C1-C4b. Proc Natl Acad Sci USA 116, 11900-11905 (2019).
15. R. Lubbers et al., Carbamylation reduces the capacity of IgG for hexamerization and complement activation. Clin Exp Immunol 200, 1-11 (2020).
16. T. H. Sharp, A. J. Koster, P. Gros, Heterogeneous MAC Initiator and Pore Structures in a Lipid Bilayer by Phase-Plate Cryo-electron Tomography. Cell Rep 15, 1-8 (2016).
17. D. C. Mastellos et al., ‘Stealth’ corporate innovation: an emerging threat for therapeutic drug development. Nat Immunol 20, 1409-1413 (2019).
18. D. V. Pedersen et al., Recruitment of properdin by bi-specific nanobodies activates the alternative pathway of complement. Mol Immunol 124, 200-210 (2020).
19. C. Seguin-Devaux et al., FHR4-based immunoconjugates direct complement-dependent cytotoxicity and phagocytosis towards HER2-positive cancer cells. Mol Oncol 13, 2531-2553 (2019).
20. J. W. Cruz et al., A novel bispecific antibody platform to direct complement activity for efficient lysis of target cells. Sci Rep 9, 12031 (2019).
21. A. Alawieh, E. F. Langley, S. Tomlinson, Targeted complement inhibition salvages stressed neurons and inhibits neuroinflammation after stroke in mice. Sci Transl Med 10 (2018).
22. S. Werneburg et al., Targeted Complement Inhibition at Synapses Prevents Microglial Synaptic Engulfment and Synapse Loss in Demyelinating Disease. Immunity 52, 167-182 e167 (2020).
23. M. Fridkis-Hareli et al., The human complement receptor type 2 (CR2)/CR1 fusion protein TT32, a novel targeted inhibitor of the classical and alternative pathway C3 convertases, prevents arthritis in active immunization and passive transfer mouse models. Mol Immunol 105, 150-164 (2019).
24. M. Fridkis-Hareli et al., Design and development of TT30, a novel C3d-targeted C3/C5 convertase inhibitor for treatment of human complement alternative pathway-mediated diseases. Blood 118, 4705-4713 (2011).

1. A bispecific antibody comprising a first antigen binding domain that binds a complement inhibitor and a second antigen binding domain that binds a target antigen.

2. A bispecific antibody according to any previous claim, wherein the complement inhibitor is selected from the group consisting of: the complement regulator factor H (FH), C4b-binding protein (C4BP), complement receptor 1 (CR1, CD35), membrane cofactor protein (MCP, CD46), decay-accelerating factor (DAF, CD55), CD59, FHL-1, mini-FH, factor I (FI) MAp44 (MBL-associated protein 1; MAP-1) and small MBL-associated protein (sMAP; also termed MAp19 or MAP-2).

3. A bispecific antibody according to any previous claim, wherein the complement inhibitor is C4BP or FH.

4. A bispecific antibody according to any previous claim, wherein the target antigen is a tissue specific antigen.

5. A bispecific antibody according to any previous claim, wherein the first antigen binding domain inhibits complement protein function, preferably the first antigen binding domain inhibits C1s or C5 complement protein function.

6. A bispecific antibody according to any previous claim, wherein the target antigen is selected from the group consisting of: Gangliosides, Acetylcholine Receptor/MUSK, Aquaporins, Amyloid Beta, TF, VCAM, NC1 domain of collagen type IV, C1q, MPO, PR3, TF, Collagen, PTM-proteins, Desmogleins, HLA-I/HLA-II antigens, and C3d.

7. A bispecific antibody according to any previous claim, further comprising an L234A, L235A (LALA) mutation and/or an L234A, L235A, P329G (LALAPG) mutation in the Fc region.

8. A bispecific antibody according to any previous claim, wherein the antigen binding domain comprises:
a) a first heavy chain variable region CDR1 comprising the amino acid sequence SEQ ID NO: 15; a heavy chain variable region CDR2 comprising the amino acid sequence SEQ ID NO: 16; a heavy chain variable region CDR3 comprising the amino acid sequence SEQ ID NO: 17 and
a first light chain variable region CDR1 comprising the amino acid sequence SEQ ID NO: 18; a light chain variable region CDR2 comprising the amino acid sequence SEQ ID NO: 19, a light chain variable region CDR2 comprising the amino acid sequence SEQ ID NO: 20 or
b) A first heavy chain variable region CDR1 comprising the amino acid sequence SEQ ID NO: 21; a heavy chain variable region CDR2 comprising the amino acid sequence SEQ ID NO: 22; a heavy chain variable region CDR3 comprising the amino acid sequence SEQ ID NO: 23 and
a first light chain variable region CDR1 comprising the amino acid sequence SEQ ID NO: 24; a light chain variable region CDR2 comprising the amino acid sequence SEQ ID NO: 25, a light chain variable region CDR2 comprising the amino acid sequence SEQ ID NO: 26.

9. A bispecific antibody according to any previous claim, wherein the antigen binding domain comprises:
a) a second heavy chain variable region CDR1 comprising the amino acid sequence SEQ ID NO: 21; a heavy chain variable region CDR2 comprising the amino acid sequence SEQ ID NO: 22; a heavy chain variable region CDR3 comprising the amino acid sequence SEQ ID NO: 23 and
a second light chain variable region CDR1 comprising the amino acid sequence SEQ ID NO: 24; a light chain variable region CDR2 comprising the amino acid sequence SEQ ID NO: 25, a light chain variable region CDR2 comprising the amino acid sequence SEQ ID NO: 26 or
a second heavy chain variable region CDR1 comprising the amino acid sequence SEQ ID NO: 15; a heavy chain variable region CDR2 comprising the amino acid sequence SEQ ID NO: 16; a heavy chain variable region CDR3 comprising the amino acid sequence SEQ ID NO: 17 and
a second light chain variable region CDR1 comprising the amino acid sequence SEQ ID NO: 18; a light chain variable region CDR2 comprising the amino acid sequence SEQ ID NO: 19, a light chain variable region CDR2 comprising the amino acid sequence SEQ ID NO: 20.

10. A bispecific antibody according to any previous claim, wherein the first and/or second antigen binding site comprises a variable heavy chain comprising amino acid SEQ ID NO: 1 and a variable light chain comprising amino acid SEQ ID NO: 2 or wherein the first and/or second antigen binding site comprises a variable heavy chain comprising amino acid SEQ ID NO: 3 and a variable light chain comprising amino acid SEQ ID NO: 4.

11. The bispecific antibody according to any previous claim for use in a method for treating or preventing a disease or condition in an individual.

12. The bispecific antibody according to any previous claim for use in the treatment of a complement mediated disease.

13. The bispecific antibody for use according to claims 11-12, wherein an additional bispecific antibody is used in the treatment of disease.

14. The bispecific antibody for use according to claims 11-13 wherein the disease is an autoimmune disease, Rheumatoid arthritis, Osteo arthritis, Psoriatic arthritis, Systemic Lupus Erythematosus, Myasthenia Gravis and Pemphigus, renal disease, atypical haemolytic uremic syndrome, Membranoproliferative glomerulonephritis, IgA nephropathy, Diabetic ANCA 3 Nephropathy, vasculitis, complement glomerulonephritis, neurological disorder, Alzheimer's disease, Parkinson's Disease, eye disease, age related macular degeneration, motor neuron disease, complement regulation disorder, paroxysmal nocturnal hemoglobinuria, arthritis, nephritis, myasthenia gravis, transplant rejection, IBMIR, cancer, infertility, pregnancy complications, or HELPP syndrome.

15. A bispecific antibody for use according to claims 11-14, wherein the bispecific antibody is administered locally.

16. A composition comprising the bispecific antibody according to claims 1-10 and at least one pharmaceutically acceptable diluent or carrier.


SEQUENCE LISTING (58 sequences)

SEQ ID NO: 1 - protein, 445 aa, Mus musculus
EVQLQQSGPELVKPGASVKISCKASGYSFTGYFIHWVKQSHVKSLEWFGRINPYNGATDYNQNFKDKAILTIDESSSTAYMEIHSLTSDDSAVYYCARGLFLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 2 - protein, 214 aa, Mus musculus
DILMTQSPSSMSVSLGDTVSITCHASQGITSNIGWLQQKPGKSFKGLIYHGTNLEDGVPSRFSGSGSGADYSLTISSLESEDFADYYCVQYAQFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 449 aa, Mus musculus
EVQLVESGGGLVKPGGSLKLSCAVSGFIFSDYYMYWVRQTPEKRLEWVATISDGGVYTYYPDSMKGRFTISRDNAKNNLYLQMSSLKSEDTAMYYCVRGDYYGNSLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 4 - protein, 219 aa, Mus musculus
DVVMTQTPLTLSITIGQPASISCKSSQSLLDGDGKTYLNWLLQRPGQSPKRLIYLVSKLDSGVPDRFTGSGSGTDFTLKISRVEAEDLGVYYCCQGTHFPWTFGRGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 451 aa, Mus musculus
DVRLQESGPGLVKPSQSLSLTCSVTGYSITNSYYWNWIRQFPGNKLEWMVYIGYDGSNNYNPSLKNRISITRDTSKNQFFLKLNSVTTEDTATYYCARATYYGNYRGFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 6 - protein, 214 aa, Mus musculus
DIRMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 457 aa, Homo sapiens
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 8 - protein, 215 aa, Homo sapiens
EIVLTQSPGTLSLSPGERATFSCRSSHSIRSRRVAWYQHKPGQAPRLVIHGVSNRASGISDRFSGSGSGTDFTLTITRVEPEDFALYYCQVYGASSYTFGQGTKLERKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 457 aa, Homo sapiens
QVQLVQSGAEVKKPGASVKVSCQASGYRFSNFVIHWVRQAPGQRFEWMGWINPYNGNKEFSAKFQDRVTFTADTSANTAYMELRSLRSADTAVYYCARVGPYSWDDSPQDNYYMDVWGKGTTVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 10 - protein, 215 aa, Homo sapiens; identical to SEQ ID NO: 8

SEQ ID NO: 11 - protein, 448 aa, Mus musculus
QVKLQESGPGLVAPSQSLSITCTVSGFSLTAYGVDWVRQPPGKGLEWLGVIWGGGRTNYNSGLMSRLSIRKDNSKSQVFLTMNSLQTDDTAKYYCVKHTNWDGGFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 12 - protein, 219 aa, Mus musculus
DVVMTQSPLSLPVSLGDQASISCRSGQGLVYSNGDTYLHWYLQKPGQSPKLLIYKVSNRFSGVPDRFSGSGSGTDFTLKISRVEAEDLGVYFCSQSTHFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 450 aa, Mus musculus
QVQLKQSGPGLVQPSQSLSLTCTVSGFSLTSYGVHWVRQPPGKGLEWLGVIWSGGSTDYNAAFISRLSIRKDNSKSQVFFKMNSLQADDTAIYYCARTFTTSTSAWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 14 - protein, 215 aa, Mus musculus
SIVMTQTPKFLLVSAGDRVTITCKASQSVSNDVAWYQQKPGQSPKLLIYYASNRYTGVPDRFTGSGYGTDFTFTISTVQAEDLAVYFCQQDYSSPPWTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 8 aa, Mus musculus
GYSFTGYF

SEQ ID NO: 16 - protein, 8 aa, Mus musculus
INPYNGAT

SEQ ID NO: 17 - protein, 8 aa, Mus musculus
ARGLFLDY

SEQ ID NO: 18 - protein, 6 aa, Mus musculus
QGITSN

SEQ ID NO: 19 - skipped

SEQ ID NO: 20 - protein, 9 aa, Mus musculus
VQYAQFPWT

SEQ ID NO: 21 - protein, 8 aa, Mus musculus
GFIFSDYY

SEQ ID NO: 22 - protein, 8 aa, Mus musculus
ISDGGVYT

SEQ ID NO: 23 - protein, 12 aa, Mus musculus
VRGDYYGNSLDY

SEQ ID NO: 24 - protein, 11 aa, Mus musculus
QSLLDGDGKTY

SEQ ID NO: 25 - skipped

SEQ ID NO: 26 - protein, 9 aa, Mus musculus
CQGTHFPWT

SEQ ID NO: 27 - protein, 9 aa, Mus musculus
GYSITNSYY

SEQ ID NO: 28 - protein, 7 aa, Mus musculus
IGYDGSN

SEQ ID NO: 29 - protein, 14 aa, Mus musculus
ARATYYGNYRGFAY

SEQ ID NO: 30 - protein, 6 aa, Mus musculus
QDISNY

SEQ ID NO: 31 - skipped

SEQ ID NO: 32 - protein, 9 aa, Mus musculus
QQGNTLPWT

SEQ ID NO: 33 - protein, 8 aa, Homo sapiens
GYRFSNFV

SEQ ID NO: 34 - protein, 8 aa, Homo sapiens
INPYNGNK

SEQ ID NO: 35 - protein, 20 aa, Homo sapiens
ARVGPYSWDDSPQDNYYMDV

SEQ ID NO: 36 - protein, 7 aa, Homo sapiens
HSIRSRR

SEQ ID NO: 37 - skipped

SEQ ID NO: 38 - protein, 9 aa, Homo sapiens
QVYGASSYT

SEQ ID NO: 39 - protein, 8 aa, Homo sapiens
GYRFSNFV

SEQ ID NO: 40 - protein, 8 aa, Homo sapiens
INPYNGNK

SEQ ID NO: 41 - protein, 31 aa, Homo sapiens
ARVGPYSWDDSPQDNYYMDVWGKGTTVIVSS

SEQ ID NO: 42 - protein, 7 aa, Homo sapiens
HSIRSRR

SEQ ID NO: 43 - skipped

SEQ ID NO: 44 - protein, 18 aa, Homo sapiens
QVYGASSYTFGQGKLERK

SEQ ID NO: 45 - protein, 8 aa, Mus musculus
GFSLTAYG

SEQ ID NO: 46 - protein, 7 aa, Mus musculus
IWGGGRT

SEQ ID NO: 47 - protein, 12 aa, Mus musculus
VKHTNWDGGFAY

SEQ ID NO: 48 - protein, 11 aa, Mus musculus
QGLVYSNGDTY

SEQ ID NO: 49 - skipped

SEQ ID NO: 50 - protein, 9 aa, Mus musculus
SQSTHFPFT

SEQ ID NO: 51 - protein, 8 aa, Mus musculus
GFSLTSYG

SEQ ID NO: 52 - protein, 7 aa, Mus musculus
IWSGGST

SEQ ID NO: 53 - protein, 14 aa, Mus musculus
ARTFTTSTSAWFAY

SEQ ID NO: 54 - protein, 6 aa, Mus musculus
QSVSND

SEQ ID NO: 55 - skipped

SEQ ID NO: 56 - protein, 10 aa, Mus musculus
QQDYSSPPWT

SEQ ID NO: 57 - protein, 19 aa, synthetic construct
  source 1..19: note = a signal peptide
MEFGLSWVFLVALLRGVQC

SEQ ID NO: 58 - protein, 6 aa, synthetic construct
LALAPG